SEQUENCE LISTING (12 sequences)

SEQ ID NO: 1 - protein, 104 aa, Artificial
  the amino acid sequence of the immune co-stimulator transmembrane domain connected with the immune co-stimulator intracellular segment of CAR3
IYIWAPLAGTCGVLLLSLVITLYCCWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 2 - protein, 104 aa, Artificial
  the amino acid sequence of the immune co-stimulator transmembrane domain connected with the immune co-stimulator intracellular segment of CAR1
IYIWAPLAGTCGVLLLSLVITLYCKRGRKKLLYIFKQPFMRPVQTTQEEDGCSCRFPEEEEGGCELCWLTKKKYSSSVHDPNGEYMFMRAVNTAKKSRLTDVTL

SEQ ID NO: 3 - protein, 103 aa, Artificial
  the amino acid sequence of the immune co-stimulator transmembrane domain connected with the immune co-stimulator intracellular segment of CAR6
IYIWAPLAGTCGVLLLSLVITLYCRRDQRLPPDAHKPPGGGSFRTPIQEEQADAHSTLAKIKRGRKKLLYIFKQPFMRPVQTTQEEDGCSCRFPEEEEGGCEL

SEQ ID NO: 4 - protein, 104 aa, Artificial
  the amino acid sequence of the immune co-stimulator transmembrane domain connected with the immune co-stimulator intracellular segment of CAR4
FWLPIGCAAFVVVCILGCILICWLKRGRKKLLYIFKQPFMRPVQTTQEEDGCSCRFPEEEEGGCELCWLTKKKYSSSVHDPNGEYMFMRAVNTAKKSRLTDVTL

SEQ ID NO: 5 - protein, 105 aa, Artificial
  the amino acid sequence of the immune co-stimulator transmembrane domain connected with the immune co-stimulator intracellular segment of CAR7
VAAILGLGLVLGLLGPLAILLALYLLRRDQRLPPDAHKPPGGGSFRTPIQEEQADAHSTLAKIKRGRKKLLYIFKQPFMRPVQTTQEEDGCSCRFPEEEEGGCEL

SEQ ID NO: 6 - protein, 105 aa, Artificial
  the amino acid sequence of the immune co-stimulator transmembrane domain connected with the immune co-stimulator intracellular segment of CAR8
VAAILGLGLVLGLLGPLAILLALYLLKRGRKKLLYIFKQPFMRPVQTTQEEDGCSCRFPEEEEGGCELRRDQRLPPDAHKPPGGGSFRTPIQEEQADAHSTLAKI

SEQ ID NO: 7 - DNA, 312 bp, Artificial
  the nucleotide sequence of the nucleic acid encoding the immune co-stimulator transmembrane domain and the immune co-stimulator intracellular segment of CAR3
atctacatctgggcgcccttggccgggacttgtggggtccttctcctgtcactggttatcaccctttactgctgttggcttacaaaaaagaagtattcatccagtgtgcacgaccctaacggtgaatacatgttcatgagagcagtgaacacagccaaaaaatctagactcacagatgtgaccctaaagcgcggccgcaagaagctgctgtacatcttcaagcagcccttcatgcgccccgtgcagaccacccaggaggaggacggctgtagctgccgcttccccgaggaggaggagggcggctgcgagctg

SEQ ID NO: 8 - DNA, 312 bp, Artificial
  the nucleotide sequence of the nucleic acid encoding the immune co-stimulator transmembrane domain and the immune co-stimulator intracellular segment of CAR1
atctacatctgggcgcccttggccgggacttgtggggtccttctcctgtcactggttatcaccctttactgcaagcgcggccgcaagaagctgctgtacatcttcaagcagcccttcatgcgccccgtgcagaccacccaggaggaggacggctgtagctgccgcttccccgaggaggaggagggcggctgcgagctgtgttggcttacaaaaaagaagtattcatccagtgtgcacgaccctaacggtgaatacatgttcatgagagcagtgaacacagccaaaaaatctagactcacagatgtgacccta

SEQ ID NO: 9 - DNA, 309 bp, Artificial
  the nucleotide sequence of the nucleic acid encoding the immune co-stimulator transmembrane domain and the immune co-stimulator intracellular segment of CAR6
atctacatctgggcgcccttggccgggacttgtggggtccttctcctgtcactggttatcaccctttactgccggcgcgaccagcgcctgccccccgacgcccacaagccccccggcggcggcagcttccgcacccccatccaggaggagcaggccgacgcccacagcaccctggccaagatcaagcgcggccgcaagaagctgctgtacatcttcaagcagcccttcatgcgccccgtgcagaccacccaggaggaggacggctgtagctgccgcttccccgaggaggaggagggcggctgcgagctg

SEQ ID NO: 10 - DNA, 312 bp, Artificial
  the nucleotide sequence of the nucleic acid encoding the immune co-stimulator transmembrane domain and the immune co-stimulator intracellular segment of CAR4
ttctggttacccataggatgtgcagcctttgttgtagtctgcattttgggatgcatacttatttgttggcttaagcgcggccgcaagaagctgctgtacatcttcaagcagcccttcatgcgccccgtgcagaccacccaggaggaggacggctgtagctgccgcttccccgaggaggaggagggcggctgcgagctgtgttggcttacaaaaaagaagtattcatccagtgtgcacgaccctaacggtgaatacatgttcatgagagcagtgaacacagccaaaaaatctagactcacagatgtgacccta

SEQ ID NO: 11 - DNA, 315 bp, Artificial
  the nucleotide sequence of the nucleic acid encoding the immune co-stimulator transmembrane domain and the immune co-stimulator intracellular segment of CAR7
gttgccgccatcctgggcctgggcctggtgctggggctgctgggccccctggccatcctgctggccctgtacctgctccggcgcgaccagcgcctgccccccgacgcccacaagccccccggcggcggcagcttccgcacccccatccaggaggagcaggccgacgcccacagcaccctggccaagatcaagcgcggccgcaagaagctgctgtacatcttcaagcagcccttcatgcgccccgtgcagaccacccaggaggaggacggctgtagctgccgcttccccgaggaggaggagggcggctgcgagctg

SEQ ID NO: 12 - DNA, 315 bp, Artificial
  the nucleotide sequence of the nucleic acid encoding the immune co-stimulator transmembrane domain and the immune co-stimulator intracellular segment of CAR8
gttgccgccatcctgggcctgggcctggtgctggggctgctgggccccctggccatcctgctggccctgtacctgctcaagcgcggccgcaagaagctgctgtacatcttcaagcagcccttcatgcgccccgtgcagaccacccaggaggaggacggctgtagctgccgcttccccgaggaggaggagggcggctgcgagctgcggcgcgaccagcgcctgccccccgacgcccacaagccccccggcggcggcagcttccgcacccccatccaggaggagcaggccgacgcccacagcaccctggccaagatc

(19) United States
(12) Patent Application Publication
(10) Pub. No.: US 2023/0235049 A1
(43) Pub. Date: Jul. 27, 2023

(54) CHIMERIC ANTIGEN RECEPTORS AND USES THEREOF

(71) Applicant: SUNSHINE LAKE PHARMA CO., LTD., Dongguan, Guangdong (CN)

(72) Inventors: Chao CHEN, Dongguan (CN); Junji DONG, Dongguan (CN); Tingting YU, Dongguan (CN); Yi ZENG, Dongguan (CN); Le XU, Dongguan (CN); Xufang WANG, Dongguan (CN); Zhiguang LI, Dongguan (CN); Shaoyan LI, Dongguan (CN); Qunrui YE, Frederick, MD (US); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 17/627,025

(22) PCT Filed: Aug. 11, 2020
(86) PCT No.: PCT/CN2020/108327

§ 371 Date: Jan. 13, 2022

(30) Foreign Application Priority Data
Aug. 12, 2019 (CN) 201910741624.1

(51) Int. Cl. C07K 16/28; C07K 14/725; C07K 14/705; C12N 15/86; A61P 35/00

(52) U.S. Cl. CPC C07K 16/2803; C07K 14/7051; C07K 14/70517; C12N 15/86; A61P 35/00; C07K 2319/03; C07K 2317/53; C12N 2740/15043

(57) ABSTRACT

An invention provides a chimeric antigen receptor. The chimeric antigen receptor includes an extracellular domain including a heavy chain variable region, a light chain variable region of a single chain antibody fragment and CD8 hinge region; a transmembrane domain including an immune co-stimulator transmembrane domain; and an intracellular domain including an immune co-stimulator intracellular segment and CD3ζ chain. According to the embodiments of the invention, the chimeric antigen receptor can specifically recognize the tumor cells expressing the specific antigen and achieve the specific killing effect against the tumor cells expressing the high specific antigen.

### CROSS-REFERENCE TO RELATED APPLICATION

This application claims the priority and benefits of Chinese Patent Application No. 201910741624.1, filed with the State Intellectual Property Office of China on Aug. 12, 2019, which is incorporated herein by reference in its entirety.

### FIELD OF THE INVENTION

The invention relates to the field of biomedicine; in particular, the invention relates to chimeric antigen receptors and uses thereof; more specifically, the invention relates to a chimeric antigen receptor, a transgenic lymphocyte expressing the chimeric antigen receptor, a construct, a lentivirus, a method for preparing the transgenic lymphocyte, a therapeutic composition for treating cancer and a method for improving the safety, effectiveness or durability of lymphocyte therapy.

### BACKGROUND OF THE INVENTION

Tumor cells may escape from immune surveillance by inducing the expression of immune inhibitory receptors, and turning off their immune response. Chimeric antigen receptor T cell (CAR-T cell) is a T cell having chimeric protein expressed on it, wherein the chimeric protein is constituted by coupling an antibody variable region which can recognize a certain tumor antigen with costimulatory structure and CD3 protein, thus CAR-T cell can specifically recognize and kill tumor target cells, and can kill tumor cells more accurately and quickly with the help of T cell cytotoxicity and antibody-targeting effect, which has achieved remarkable results in haematological malignancy treatment, and it is a rapidly developing individualized tumor treatment program. However, there are some problems in the application of CAR-T. Improving and solving the existing problems in CAR-T treatment is the key to the development of CAR-T treatment in the future.

### SUMMARY OF THE INVENTION

The disclosure of the present invention is based on the inventors' discovery and recognization of the following fact and problems:

At present in the treatment of chimeric antigen receptor gene modification T cell therapy (CAR-T), there are many problems such as high recurrence rate, high cytokine inflammation, poor persistence, and sometimes even neurotoxicity, and so on.

An objective of the present invention is aimed to solve one of technical problems mentioned above. Based on the prior art of CAR-T, the inventors of the present application have designed a novel immune co-stimulator tandem structure of chimeric antigen receptor (CAR). It is surprised that CAR-T cells having this tandem structure CAR can effectively reduce the self release of CAR-T cell inflammatory factors, increase the proportion of central memory T cells (T_{CM}), and can simulate the immune response and activate the function of normal T cells, which is expected to solve the existing problems in CAR-T treatment in prior art.

In a first aspect of the invention, the invention provides a chimeric antigen receptor. According to the embodiments of the invention, the chimeric antigen receptor comprises an extracellular domain including a heavy chain variable region, a light chain variable region of a single chain antibody and CD8 hinge region; a transmembrane domain including an immune co-stimulator transmembrane domain; and an intracellular domain including an immune co-stimulator intracellular segment and CD3ζ chain (CD3 zeta). According to the embodiments of the invention, the chimeric antigen receptor can specifically recognize tumor cells expressing the specific antigen and achieve the specific killing effect against the tumor cells highly expressing the specific antigen.

According to the embodiments of the invention, the above chimeric antigen receptor further comprises at least one of the following additional technical features:

According to the embodiments of the invention, the immune co-stimulator transmembrane domain is CD8 transmembrane domain; the immune co-stimulator intracellular segment is 4-1BB intracellular segment and ICOS or OX-40 intracellular segment.

According to the embodiments of the invention, the immune co-stimulator transmembrane domain is ICOS transmembrane domain; the immune co-stimulator intracellular segment is 4-1BB intracellular segment and ICOS intracellular segment.

According to the embodiments of the invention, the immune co-stimulator transmembrane domain is OX40 transmembrane domain; the immune co-stimulator intracellular segment is 4-1BB intracellular segment and OX40 intracellular segment.

Through experiments, the inventors found that the expression titer of chimeric antigen receptor, the killing effect of immune cells expressing chimeric antigen receptor and the release of cytokines were significantly affected, while different immune costimulator transmembrane domain is combined with different immune costimulators intracellular segment.

According to the embodiments of the invention, wherein the immune co-stimulator transmembrane domain is CD8 transmembrane domain, the immune co-stimulator intracellular segment is 4-1BB intracellular segment and ICOS intracellular segment, the N-terminus of the ICOS intracellular segment is linked to the C-terminus of the CD8 transmembrane domain, the C-terminus of the ICOS intracellular segment is linked to the N-terminus of the 4-1BB intracellular segment, the C-terminus of the 4-1BB intracellular segment is linked to the N-terminus of the CD3ζ chain. The inventors found that while the immune co-stimulator transmembrane domain in chimeric antigen receptor is linked to the intracellular segment in the above order, the chimeric antigen receptor had a high expression titer in adenovirus, and the immune cells expressing the chimeric antigen receptor had a significant specific killing effect against the tumor cells expressing CD19, and the non-specific killing effect and cell inflammatory factor response were weak.

According to the embodiments of the invention, wherein the immune co-stimulator transmembrane domain is CD8 transmembrane domain, the immune co-stimulator intracellular segment is 4-1BB intracellular segment and OX-40 intracellular segment, the N-terminus of the OX-40 intracellular segment is linked to the C-terminus of the CD8 transmembrane domain, the C-terminus of the OX-40 intracellular segment is linked to the N-terminus of the 4-1BB intracellular segment, the C-terminus of the 4-1BB intracellular segment is linked to the N-terminus of the CD3ζ chain. The inventors found that while the immune co-stimulator transmembrane domain in chimeric antigen receptor is linked to the intracellular segment in the above order, the cytokine INF γ release was decreased on the premise that the specific killing effect of immune cells expressing the chimeric antigen receptor against tumor cells expressing CD19 is equivalent to that of second generation CAR-T.

According to the embodiments of the invention, wherein the immune co-stimulator transmembrane domain is ICOS transmembrane domain, the immune co-stimulator intracellular segment is 4-1BB intracellular segment and ICOS intracellular segment, the N-terminus of the 4-1BB intracellular segment is linked to the C-terminus of the ICOS transmembrane domain, the C-terminus of the 4-1BB intracellular segment is linked to the N-terminus of the ICOS intracellular segment, the C-terminus of the ICOS intracellular segment is linked to the N-terminus of the CD3ζ chain. The inventors found that while the immune co-stimulator transmembrane domain in chimeric antigen receptor is linked to the intracellular segment in the above order, the specific killing effect of immune cells expressing the chimeric antigen receptor against tumor cells expressing CD19 is significantly better than that of second generation CAR-T.

According to the embodiments of the invention, wherein the immune co-stimulator transmembrane domain connected with the immune co-stimulator intracellular segment has any one amino acid sequence of SEQ ID NO:1 to 6.

(SEQ ID NO: 1)
IYIWAPLAGTCGVLLLSLVITLYCCWLTKKKYSSSVHDPNGEYMFMRAV
NTAKKSRLTDVTLKRGRKKLLYIFKQPFMRPVQTTQEEDGCSCRFPEEE
EGGCEL.
(SEQ ID NO: 2)
IYIWAPLAGTCGVLLLSLVITLYCKRGRKKLLYIFKQPFMRPVQTTQEE
DGCSCRFPEEEEGGCELCWLTKKKYSSSVHDPNGEYMFMRAVNTAKKSR
LTDVTL.
(SEQ ID NO: 3)
IYIWAPLAGTCGVLLLSLVITLYCRRDQRLPPDAHKPPGGGSFRTPIQE
EQADAHSTLAKIKRGRKKLLYIFKQPFMRPVQTTQEEDGCSCRFPEEEE
GGCEL.
(SEQ ID NO: 4)
FWLPIGCAAFVVVCILGCILICWLKRGRKKLLYIFKQPFMRPVQTTQEE
DGCSCRFPEEEEGGCELCWLTKKKYSSSVHDPNGEYMFMRAVNTAKKSR
LTDVTL.
(SEQ ID NO: 5)
VAAILGLGLVLGLLGPLAILLALYLLRRDQRLPPDAHKPPGGGSFRTPI
QEEQADAHSTLAKIKRGRKKLLYIFKQPFMRPVQTTQEEDGCSCRFPEE
EEGGCEL.
(SEQ ID NO: 6)
VAAILGLGLVLGLLGPLAILLALYLLKRGRKKLLYIFKQPFMRPVQTTQ
EEDGCSCRFPEEEEGGCELRRDQRLPPDAHKPPGGGSFRTPIQEEQADA
HSTLAKI.

Wherein the chimeric antigen receptor having the amino acid sequence of SEQ ID NO:1 was referred to as CAR3 in this application (the structure from N-terminus to C-terminus may be expressed as: scFv-CD8hinge+CD8TM-ICOS-4-1BB-CD3 zeta, wherein hinge is hinge domain, TM is transmembrane domain), the chimeric antigen receptor having the amino acid sequence of SEQ ID NO:2 was referred to as CAR1 in this application (the structure from N-terminus to C-terminus may be expressed as: scFv-CD8hinge+CD8TM-4-1BB-ICOS-CD3 zeta), the chimeric antigen receptor having the amino acid sequence of SEQ ID NO:3 was referred to as CAR6 in this application (the structure from N-terminus to C-terminus may be expressed as: scFv-CD8hinge+CD8TM-OX40-4-1BB-CD3 zeta), the chimeric antigen receptor having the amino acid sequence of SEQ ID NO:4 was referred to as CAR4 in this application (the structure from N-terminus to C-terminus may be expressed as: scFv-CD8hinge+ICOSTM-4-1BB-ICOS-CD3 zeta), the chimeric antigen receptor having the amino acid sequence of SEQ ID NO:5 was referred to as CART in this application (the structure from N-terminus to C-terminus may be expressed as: scFv-CD8hinge+OX40TM-OX40-4-1BB-CD3 zeta), the chimeric antigen receptor having the amino acid sequence of SEQ ID NO:6 was referred to as CAR8 in this application (the structure from N-terminus to C-terminus may be expressed as: scFv-CD8hinge+OX40TM-4-1BB-OX40-CD3 zeta).

According to the embodiments of the invention, wherein the antigen comprises at least one selected from CD19, CD20, CD123, GPC3, MUC-1, GD2, BCMA, HER2, EGFR, VEGFR, cMet, MSLN, EGFRvIII and Claudin 18.2.

In a second aspect of the invention, the invention provides a construct. According to the embodiments of the invention, wherein the construct comprises a nucleic acid molecule encoding the chimeric antigen receptor of the invention. The construct according to the embodiment of the invention is introduced into a receptor cell, and the chimeric antigen receptor described above can be efficiently expressed in the receptor cell.

According to the embodiments of the invention, the above construct further comprises at least one of the following additional technical features:

according to the embodiments of the invention, the construct further comprises a promoter, wherein the promoter is operably connected with the nucleic acid molecule;

according to the embodiments of the invention, wherein the promoter comprises at least one selected from CMV, EF-1α, RSV;

according to the embodiments of the invention, the construct is nonpathogenic virus;

according to the embodiments of the invention, wherein the virus is selected from retroviruses, lentiviruses and adenovirus related viruses.

In a third aspect of the invention, the present invention provides a lentivirus. According to the embodiments of the invention, wherein the lentivirus carries a nucleic acid molecule having a nucleotide sequence of SEQ ID NO: 7 to 12. The expression of the chimeric antigen receptor in the receptor cells can be realized by using lentivirus to infect receptor cell according to the embodiment of the invention, such as activated T-lymphocytes, and the cells expressing the chimeric antigen receptor, such as T lymphocytes, can achieve specific killing against tumor cells with high expression of specific antigens, moreover, the non-specific killing and cell inflammatory factor response are weak, which overcomes the existing treatment problems of CAR-T cells.

(SEQ ID NO: 7)
ATCTACATCTGGGCGCCCTTGGCCGGGACTTGTGGGGTCCTTCTCCTGT
CACTGGTTATCACCCTTTACTGCTGTTGGCTTACAAAAAAGAAGTATTC
ATCCAGTGTGCACGACCCTAACGGTGAATACATGTTCATGAGAGCAGTG
AACACAGCCAAAAAATCTAGACTCACAGATGTGACCCTAAAGCGCGGCC
GCAAGAAGCTGCTGTACATCTTCAAGCAGCCCTTCATGCGCCCCGTGCA
GACCACCCAGGAGGAGGACGGCTGTAGCTGCCGCTTCCCCGAGGAGGAG
GAGGGCGGCTGCGAGCTG.
(SEQ ID NO: 8)
ATCTACATCTGGGCGCCCTTGGCCGGGACTTGTGGGGTCCTTCTCCTGT
CACTGGTTATCACCCTTTACTGCAAGCGCGGCCGCAAGAAGCTGCTGTA
CATCTTCAAGCAGCCCTTCATGCGCCCCGTGCAGACCACCCAGGAGGAG
GACGGCTGTAGCTGCCGCTTCCCCGAGGAGGAGGAGGGCGGCTGCGAGC
TGTGTTGGCTTACAAAAAAGAAGTATTCATCCAGTGTGCACGACCCTAA
CGGTGAATACATGTTCATGAGAGCAGTGAACACAGCCAAAAAATCTAGA
CTCACAGATGTGACCCTA.
(SEQ ID NO: 9)
ATCTACATCTGGGCGCCCTTGGCCGGGACTTGTGGGGTCCTTCTCCTGT
CACTGGTTATCACCCTTTACTGCCGGCGCGACCAGCGCCTGCCCCCCGA
CGCCCACAAGCCCCCCGGCGGCGGCAGCTTCCGCACCCCCATCCAGGAG
GAGCAGGCCGACGCCCACAGCACCCTGGCCAAGATCAAGCGCGGCCGCA
AGAAGCTGCTGTACATCTTCAAGCAGCCCTTCATGCGCCCCGTGCAGAC
CACCCAGGAGGAGGACGGCTGTAGCTGCCGCTTCCCCGAGGAGGAGGAG
GGCGGCTGCGAGCTG.
(SEQ ID NO: 10)
TTCTGGTTACCCATAGGATGTGCAGCCTTTGTTGTAGTCTGCATTTTGG
GATGCATACTTATTTGTTGGCTTAAGCGCGGCCGCAAGAAGCTGCTGTA
CATCTTCAAGCAGCCCTTCATGCGCCCCGTGCAGACCACCCAGGAGGAG
GACGGCTGTAGCTGCCGCTTCCCCGAGGAGGAGGAGGGCGGCTGCGAGC
TGTGTTGGCTTACAAAAAAGAAGTATTCATCCAGTGTGCACGACCCTAA
CGGTGAATACATGTTCATGAGAGCAGTGAACACAGCCAAAAAATCTAGA
CTCACAGATGTGACCCTA.
(SEQ ID NO: 11)
GTTGCCGCCATCCTGGGCCTGGGCCTGGTGCTGGGGCTGCTGGGCCCCC
TGGCCATCCTGCTGGCCCTGTACCTGCTCCGGCGCGACCAGCGCCTGCC
CCCCGACGCCCACAAGCCCCCCGGCGGCGGCAGCTTCCGCACCCCCATC
CAGGAGGAGCAGGCCGACGCCCACAGCACCCTGGCCAAGATCAAGCGCG
GCCGCAAGAAGCTGCTGTACATCTTCAAGCAGCCCTTCATGCGCCCCGT
GCAGACCACCCAGGAGGAGGACGGCTGTAGCTGCCGCTTCCCCGAGGAG
GAGGAGGGCGGCTGCGAGCTG.
(SEQ ID NO: 12)
GTTGCCGCCATCCTGGGCCTGGGCCTGGTGCTGGGGCTGCTGGGCCCCC
TGGCCATCCTGCTGGCCCTGTACCTGCTCAAGCGCGGCCGCAAGAAGCT
GCTGTACATCTTCAAGCAGCCCTTCATGCGCCCCGTGCAGACCACCCAG
GAGGAGGACGGCTGTAGCTGCCGCTTCCCCGAGGAGGAGGAGGGCGGCT
GCGAGCTGCGGCGCGACCAGCGCCTGCCCCCCGACGCCCACAAGCCCCC
CGGCGGCGGCAGCTTCCGCACCCCCATCCAGGAGGAGCAGGCCGACGCC
CACAGCACCCTGGCCAAGATC.

Wherein the nucleic acid having the nucleotide sequence of SEQ ID NO:7 encodes the immune co-stimulator transmembrane domain and the immune co-stimulator intracellular segment of the chimeric antigen receptor CAR3, the nucleic acid having the nucleotide sequence of SEQ ID NO:8 encodes the immune co-stimulato transmembrane domain and the immune co-stimulator intracellular segment of the chimeric antigen receptor CAR1, the nucleic acid having the nucleotide sequence of SEQ ID NO:9 encodes the immune co-stimulator transmembrane domain and the immune co-stimulator intracellular segment of the chimeric antigen receptor CARE, the nucleic acid having the nucleotide sequence of SEQ ID NO:10 encodes the immune co-stimulator transmembrane domain and the immune co-stimulator intracellular segment of the chimeric antigen receptor CAR4, the nucleic acid having the nucleotide sequence of SEQ ID NO:11 encodes the immune co-stimulator transmembrane domain and the immune costimulator intracellular segment of the chimeric antigen receptor CART, the nucleic acid having the nucleotide sequence of SEQ ID NO:12 encodes the immune co-stimulator transmembrane domain and the immune co-stimulator intracellular segment of the chimeric antigen receptor CARE.

In a forth aspect of the invention, the present invention provides a transgenic lymphocyte. According to the embodiments of the invention, the transgenic lymphocyte expresses the chimeric antigen receptor, or the transgenic lymphocyte is obtained by introducing the construct or the lentivirus into a lymphocyte. The transgenic lymphocyte according to the embodiment of the invention can specifically recognize and kill the tumor cells with high expression of specific antigen, and the non-specific killing and cell inflammatory factor response are weak.

According to the embodiments of the invention, the transgenic lymphocyte further comprises at least one of the following additional technical features:

according to the embodiments of the invention, the transgenic lymphocyte is a CD3⁺T cell;

according to the embodiments of the invention, the transgenic lymphocyte is a natural killer cell;

according to the embodiments of the invention, the transgenic lymphocyte is a natural killer T cell.

In a fifth aspect of the invention, the present invention provides a method for preparing the transgenic lymphocyte as described above. According to the embodiments of the invention, the method comprises introducing the above construct or the above lentivirus into a lymphocyte.

In the sixth aspect of the invention, the present invention provides a therapeutic composition for treating cancer. According to the embodiments of the invention, the therapeutic composition comprises the above construct, the above lentivirus, the above transgenic lymphocyte. The therapeutic composition according to the embodiment of the invention has significant specific killing effect against tumor cells, and overcomes the problems of high recurrence rate, high cytokine inflammation and poor persistence in the prior art CAR-T therapy.

According to the embodiments of the invention, the aforementioned therapeutic composition further comprises at least one of the following additional technical features:

according to the embodiments of the invention, the cancer comprises a hematopoietic malignancy, a gastrointestinal cancer, a glioma, a lung cancer, a liver cancer, a pancreatic cancer.

In the seventh aspect of the invention, the present invention provides a method to improve the safety, effectiveness or permanent of lymphocyte therapy. According to the embodiments of the invention, the transgenic lymphocyte expresses the aforementioned chimeric antigen receptor.

In the seventh aspect of the invention, the present invention provides the aforementioned chimeric antigen receptor for use in improving the safety, effectiveness or persistence of lymphocyte therapy. According to the embodiments of the invention, the transgenic lymphocyte expresses the aforementioned chimeric antigen receptor.

In the seventh aspect of the invention, the present invention provides the use of the aforementioned chimeric antigen receptor in improving the safety, effectiveness or persistence of lymphocyte therapy. According to the embodiments of the invention, the transgenic lymphocyte expresses the aforementioned chimeric antigen receptor.

The chimeric antigen receptor and the lymphocyte expressing the chimeric antigen receptor according to the embodiment of the invention have the following advantages:

1. effectively reducing CAR-T self-activation and factor self-release;

2. increasing the proportion of central memory T cells (T_{CM}) and effector memory T cells (T_{EM}) subsets of CAR-T cells, reducing the recurrence rate of cancer, and simulating the immune-activated proliferative function of normal T cells.

### BRIEF DESCRIPTION OF THE DRAWINGS

FIG. 1 is the result diagram of virus packaging of CAR with different structures and their positive expression rate according to the embodiment of the invention;

FIG. 2 is the result diagram of the cytotoxicity of T cells carrying CAR with different structures according to the embodiment of the invention;

FIG. 3 is the result diagram of INF-r release of T cells carrying CAR with different structures according to the embodiment of the invention in cytotoxicity experiments;

FIG. 4 is the result diagram of CAR positive rate of T cells carrying CAR with different structures according to the embodiment of the invention after tumor cell stimulation

FIG. 5 is the result diagram of cytokine self-release of T cells carrying CAR with different structures according to the embodiment of the invention when co-cultured with non-tumor cells

FIG. 6 is the effect diagram of specific and nonspecific killing of CAR-T 3 after CAR-T 3 according to the embodiment of the invention was co-cultured with effector cells;

FIG. 7 is the effect diagram of CAR positive rate after CAR-T 3 according to the embodiment of the invention was co-cultured with effector cells; and

FIG. 8 is the result diagram of cytokine release of anti-Claudin 18.2 CAR-T with different structures according to the embodiment of the invention when cultured alone or co-cultured with MGC-803-18.1 for 24 h.

### SPECIFIC IMPLEMENTATION

Embodiments of the present invention are described in detail below, the example of the embodiments is shown in the drawings. The embodiments described below are exemplary by referring to the drawings, which are merely to explain the present invention, but not to limit the scope of the present invention.

### DETAILED DESCRIPTION

Unless otherwise defined, all technical and scientific terms used herein have the same meaning as commonly understood by those skilled in the art to which the invention pertains. Although any methods and materials similar or equivalent to those described herein can be used in the practice for testing of the present invention, the preferred materials and methods are described herein. In describing and claiming the present invention, the following terminology will be used.

The present invention relates to a chimeric antigen receptor (CAR), CARs are molecules that combine antibody-based specificity for a desired antigen (e.g., tumor antigen) with a T cell receptor-activating intracellular domain to generate a chimeric protein that exhibits a specific anti-tumor cellular immune activity.

T cells expressing a CAR are referred to herein as CAR T cells or CAR modified T cells.

In one embodiment, the CAR of the present invention includes an extracellular domain having an antigen recognition domain, a transmembrane domain, and an intracellular domain.

A “lentivirus” as used herein refers to a genus of the Retroviridae family Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

CARs disclosed in the embodiments of the present invention (including functional portions and functional variants) were obtained by methods known in the art. CAR may be prepared by any suitable method of preparing proteins or polypeptides. Suitable methods for de novo synthesis of peptides and proteins are described in references, for example, Chan et al, Fmoc Solid Phase Peptide Synthesis, Oxford University Press, Oxford, United Kingdom, 2000; Peptide and Protein DrugAnalysis, Reid, R., Marcel Dekker Inc., 2000; Epitope Mapping, Westwood et al, Oxford University Press, Oxford, United Kingdom, 2001; U.S. Pat. No. 5,449,752. In addition, polypeptides and proteins may be produced recombinantly using standard recombinant methods and based on the nucleic acids described herein. See, for example, Sambrook et al., Molecular Cloning: A Laboratory Manual, 3rd Edition, Cold Spring HarborPress, Cold Spring Harbor, N.Y. 2001; and Ausubel et al., Current Protocols in MolecularBiology, Greene Publishing Associates and John Wiley&Sons, N Y, 1994. In addition, some CARs of the present invention (including functional portions and functional variants thereof) can be isolated and/or purified from origin such as plants, bacteria, insects, mammals such as rats, humans, and the like. Separation and purification methods are well known in the art. Optionally, the CAR described herein (including functional portions and functional variants thereof) may be synthesized by a commercial company like Synpep (Dublin. Calif.). Peptide Technologies Corp. (Gaithersburg. Md.) and Multiple Peptide Systems (San Diego, Calif.). In this aspect, the CAR of the present invention can be synthesized, recombined, isolated and/or purified.

Methods of testing antibodies for the ability to bind to any functional portion of the inventive CAR are known in the art and include any antibody-antigen binding assay, such as, radioimmunoassay (RIA), ELISA, Western blot, immunoprecipitation, and competitive inhibition assays (see, e.g., Janeway et al., infra, and U.S. Patent Application Publication No. 2002/0197266 A1).

The invention also includes the functional variants of the CAR of the invention described herein within the scope of the invention. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98%), about 99% or more identical in amino acid sequence to the parent CAR.

A functional variant can, for example, comprise the amino acid sequence of the parent CAR with at least one conservative amino acid substitution. Alternatively, or additionally, a functional variant can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Tip, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Tip, and Tyr), etc.

The CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenyl serine β-hydroxyphenylalanine, phenylglycine, -naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

An embodiment of the invention also provides antigen binding portions of any of the antibodies described herein. The antigen binding portion can be any portion that has at least one antigen binding site, such as Fab, F(ab′)2, dsFv, sFv, diabodies, and triabodies. A single-chain variable region fragment (sFv) antibody fragment, which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology (see, e.g., Reiter et al., Protein Engineering, 7, 697-704 (1994)). Antibody fragments of the invention, however, are not limited to these exemplary types of antibody fragments.

Further provided by an embodiment of the invention is a nucleic acid comprising a-nucleotide sequence encoding any of the CARs described herein (including functional portions and functional variants thereof). The nucleic acids of the invention may comprise a nucleotide sequence encoding any of extracellular domains, transmembrane domains and/or intracellular domains described herein.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR and which may or may not be translated upon expression of the nucleic acid by a host cell.

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the invention can be incorporated into a recombinant expression vector or a construct. In this regard, an embodiment of the invention provides recombinant expression vectors or construct comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell, the genetically modified oligonucleotide or polynucleotide construct allows the host cell to express mRNA, protein, polypeptide or peptide. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNMI 149, also can be used. Examples of plant expression vectors include pBIO1, pBI101.2, pBI101.3, pBI121 and pBIN19 (Clontech). Examples of animal expression vectors include pEU-Cl, pMAM, and pMAMneo (Clontech). The recombinant expression vector may be a viral vector, e.g., a retroviral vector or a lentiviral vector. In some embodiments, the vector can be a transposon.

A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al., Gene, 13: 97 (1981)). Transfection methods include calcium phosphate co-precipitation (see, e.g., Graham et al.,), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al., Nature, 327: 70-73 (1987)).

In an embodiment, the recombinant expression vectors of the invention can be prepared using standard recombinant DNA techniques described in, for example, Sambrook et al., and Ausubel et al. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma vims, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

Included in the scope of the invention are conjugates, e.g., bioconjugates, comprising any of the inventive CARs (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

An embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4+/CD8+double positive T cells, CD4+ helper T cells, e.g., Thi and Th2 cells, CD8+ T cells (e.g., cytotoxic T cells), tumor infiltrating cells, memory T cells, naive T cells, and the like. The T cell may be a CD8+ T cell or a CD4+ T cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

CARs (including functional portions and variants thereof), nucleic acids, recombinant expression vectors, host cells (including populations thereof), and antibodies (including antigen binding portions thereof), all of which are collectively referred to as “inventive CAR materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is purer than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The inventive CAR materials can be formulated into a composition, such as a pharmaceutical composition. In this regard, an embodiment of the invention provides a pharmaceutical composition comprising any of the CARs, functional portions, functional variants, nucleic acids, expression vectors, host cells (including populations thereof), and antibodies (including antigen binding portions thereof), and a pharmaceutically acceptable carrier. The inventive pharmaceutical compositions containing any of the inventive CAR materials can comprise more than one inventive CAR material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

The inventive CAR materials can be provided in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive CAR material, as well as by the particular method used to administer the inventive CAR material. Accordingly, there are a variety of suitable formulations of the pharmaceutical composition of the invention. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight c-f the total composition.

The concentration of inventive CAR material in the pharmaceutical formulations can vary, e.g., from less than about 1%, usually at or at least about 10%, to as much as about 20% to about 50% or more by weight, and can be selected primarily by fluid volumes, and viscosities, in accordance with the particular mode of administration selected.

Methods for preparing administrable (e.g., parenterally administrable) compositions are known or apparent to those skilled in the art and are described in more detail in, for example, Remington: The Science and Practice of Pharmacy, Lippincott Williams & Wilkins; 21 st ed. (May 1, 2005).

The following formulations for oral, aerosol, parenteral (e.g., subcutaneous, intravenous, intraarterial, intramuscular, intradermal, interperitoneal, and intrathecal), and topical administration are merely exemplary and are in no way limiting. More than one route can be used to administer the inventive CAR materials, and in certain instances, a particular route can provide a more immediate and more effective response than another route.

Formulations suitable for oral administration can comprise or consist of (a) liquid solutions, such as an effective amount of the inventive CAR material dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or softshelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the inventive CAR material in a flavor, usually sucrose and acacia or tragacanth, as well as pastilles comprising the inventive CAR material in an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The inventive CAR material can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable fatty acid salts for use in parenteral formulations include fatty alkali metal, ammonium, and tri ethanol amine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain, for example, from about 0.5% to about 25% by weight of the inventive CAR material in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with an embodiment of the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

Topical formulations, including those that are useful for transdermal drug release, are well known to those of skill in the art and are suitable in the context of embodiments of the invention for application to skin. The inventive CAR material, alone or in combination with other suitable components, can be made into aerosol formulations to be administered via inhalation. These aerosol formulations can be placed into pressurized acceptable propellants, such as dichlorodifiuoromethane, propane, nitrogen, and the like. They also may be formulated as pharmaceuticals for non-pressured preparations, such as in a nebulizer or an atomizer. Such spray formulations also may be used to spray mucosa.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive CAR materials in each or various rounds of administration. By way of example and not intending to limit the invention, the dose of the inventive CAR material can be about 0.001 to about 1000 mg/kg body weight of the subject being treated/day, from about 0.01 to about 10 mg/kg body weight/day, about 0.01 mg to about 1 mg/kg body weight/day. When the inventive CAR material is a host cell, an exemplary dose of host cells may be a minimum of one million cells (1 mg cells/dose). When the inventive CAR material is a nucleic acid packaged in a virus, an exemplary dose of virus may be 1 ng/dose.

For purposes of the invention, the amount or dose of the inventive CAR material administered should be sufficient to effect a therapeutic or prophylactic response in the subject or animal over a reasonable time frame. For example, the dose of the inventive CAR material should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular inventive CAR material and the condition of the animal (e.g., human), as well as the body weight of the animal (e.g., human) to be treated.

For purposes of the invention, an assay, which comprises, for example, comparing the extent to which target cells are lysed and/or IFN-γ is secreted by T cells expressing the inventive CAR upon administration of a given dose of such T cells to a mammal, among a set of mammals of which is each given a different dose of the T cells, could be used to determine a starting dose to be administered to a mammal. The extent to which target cells are lysed and/or IFN-γ is secreted upon administration of a certain dose can be assayed by methods known in the art.

In addition to the afore described pharmaceutical compositions, the inventive CAR materials can be formulated as inclusion complexes, such as cyclodextrin inclusion complexes, or liposomes. Liposomes can serve to target the inventive CAR materials to a particular tissue. Liposomes can serve to target the inventive CAR materials to a particular tissue. Liposomes also can be used to increase the half-life of the inventive CAR materials. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The delivery systems useful in the context of embodiments of the invention may include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polycaprolactones, polyesteramides, polyortho esters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

One of ordinary skill in the art will readily appreciate that the inventive CAR materials of the invention can be modified in any number of ways, such that the therapeutic or prophylactic efficacy of the inventive CAR materials is increased through the modification. For instance, the inventive CAR materials can be conjugated either directly or indirectly through a linker to a targeting moiety. The practice of conjugating compounds, e.g., inventive CAR materials, to targeting moieties is known in the art. See, for instance, Wadwa et al., J. Drug Targeting 3: 1 1 1 (1995) and U.S. Pat. No. 5,087,616.

Alternatively, the inventive CAR materials can be modified into a depot form, such that the manner in which the inventive CAR materials is released into the body to which it is administered is controlled with respect to time and location within the body (see, for example, U.S. Pat. No. 4,450,150).

Depot forms of inventive CAR materials can be, for example, an implantable composition comprising the inventive CAR materials and a porous or non-porous material, such as a polymer, wherein the inventive CAR materials are encapsulated by or diffused throughout the material and/or degradation of the non-porous material. The depot is then implanted into the desired location within the body and the inventive CAR materials are released from the implant at a predetermined rate.

When the inventive CAR materials are administered with one or more additional therapeutic agents, one or more additional therapeutic agents can be coadministered to the mammal. By “coadministering” is meant administering one or more additional therapeutic agents and the inventive CAR materials sufficiently close in time such that the inventive CAR materials can enhance the effect of one or more additional therapeutic agents, or vice versa. In this regard, the inventive CAR materials can be administered first and the one or more additional therapeutic agents can be administered second, or vice versa. Alternatively, the inventive CAR materials and the one or more additional therapeutic agents can be administered simultaneously. For purposes of the inventive methods, wherein host cells or populations of cells are administered, the cells can be cells that are allogeneic or autologous to the mammal.

It is contemplated that the inventive pharmaceutical compositions, CARs, nucleic acids, recombinant expression vectors, host cells, or populations of cells can be used in methods of treating or preventing a disease in a mammal. Without being bound to a particular theory or mechanism, the inventive CARs have biological activity, e.g., ability to recognize antigen, such that the CAR when expressed by a cell is able to mediate an immune response against the cell expressing the antigen, for which the CAR is specific. In this regard, an embodiment of the invention provides a method of treating or preventing cancer in a mammal, comprising administering to the mammal the CARs, the nucleic acids, the recombinant expression vectors, the host cells, the population of cells, the antibodies and/or the antigen binding portions thereof, and/or the pharmaceutical compositions of the invention in an amount effective to treat or prevent cancer in the mammal.

An embodiment of the invention further comprises lymphodepleting the mammal prior to administering the inventive CAR materials. Examples of lymphodepletion include, but may not be limited to, nonmyeloablative lymphodepleting chemotherapy, myeloablative lymphodepleting chemotherapy, total body irradiation, etc.

For purposes of the inventive methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, chronic lymphocytic leukemia, acute lymphocytic cancer, acute myeloid leukemia, B-chronic lymphocytic leukemia, hairy cell leukemia, acute lymphocytic leukemia (ALL), and Burkitt's lymphoma). Preferably, the cancer is characterized by the expression of CD22.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the invention provides a use of the inventive CARs, nucleic acids, recombinant expression vectors, host cells, populations of cells, antibodies, or antigen binding portions thereof, or pharmaceutical compositions, for the treatment or prevention of cancer in a mammal.

In addition, CAR function can be evaluated by measurement of cellular cytoxicity, as described in Zhao et al., J. Immunol, 174: 4415-4423 (2005).

The following examples further illustrate the invention but, of course, should not be construed as in any way limiting its scope.

### Example 1 Virus Packaging of Chimeric Antigen Receptor

1, Synthesis of CAR having different structures, the structures of CAR with signal peptide are shown in Table 1 below.

TABLE 1

| No. | Abbreviation | CAR with signal peptide |
|---|---|---|
| 1 | CAR1 | Signal peptide -scFv -CD8hinge + |
| | | CD8TM-4-1BB-ICOS-CD3 zeta |
| 2 | CAR2 | Signal peptide-scFv -CD8hinge + |
| | | ICOSTM-ICOS-CD3 zeta |
| 3 | CAR3 | Signal peptide-scFv -CD8hinge + |
| | | CD8TM-ICOS-4-1BB -CD3 zeta |
| 4 | CAR4 | Signal peptide-scFv -CD8hinge + |
| | | ICOSTM-4-1BB-ICOS-CD3 zeta |
| 5 | CAR5 | Signal peptide- scFv -CD8hinge + |
| | | ICOSTM-ICOS-4-1BB -CD3 zeta |
| 6 | CAR6 | Signal peptide-scFv -CD8hinge + |
| | | CD8TM-OX40-4-1BB -CD3 zeta |
| 7 | CAR7 | Signal peptide-scFv -CD8hinge + |
| | | OX40TM-OX40-4-1BB -CD3 zeta |
| 8 | CAR8 | Signal peptide-scFv -CD8hinge + |
| | | OX40TM-4-1BB-OX40-CD3 zeta |
| 9 | CAR9 | Signal peptide-scFv CD8hinge + |
| | | CD8TM-4-1BB-OX40-CD3 zeta |
| 10 | CAR10 | Signal peptide-scFv CD8hinge + |
| (Second-generation | | CD8TM-4-1BB-CD3 zeta |
| CAR control) | | |

Wherein the amino acid sequence of each part of the CAR with the signal peptide and the corresponding nucleotide sequence of the encoding nucleic acid are as follows:

Nucleotide sequence of the signal peptide (GL):
Atgggcgtcaaggtcctgttcgccctgatctgcatcgccgtcgccgagg
cc
Amino acid sequence of the signal peptide (GL):
MGVKVLFALICIAVAEA
Nucleotide sequence of CD8 hinge:
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT
Amino acid sequence of CD8 hinge:
TTTPAPRPPTPAPTIASQPLSLRPEACRPAAGGAVHTRGLDFACD
Nucleotide sequence of CD8 trans-membrane (TM):
ATCTACATCTGGGCGCCCTTGGCCGGGACTTGTGGGGTCCTTCTCCTGT
CACTGGTTATCACCCTTTACTGC
Amino acid sequence of CD8 trans-membrane (TM):
IYIWAPLAGTCGVLLLSLVITLYC
Nucleotide sequence of ICOS trans-membrane (TM)
ttctggttacccataggatgtgcagcctttgttgtagtctgcattttgg
gatgcatacttatttgttggctt
Amino acid sequence of ICOS trans-membrane (TM)
FWLPIGCAAFVVVCILGCILICWL
Nucleotide sequence of OX40 trans-membrane (TM):
gttgccgccatcctgggcctgggcctggtgctggggctgctgggccccc
tggccatcctgctggccctgtacctgctc
Amino acid sequence of OX40 trans-membrane (TM):
VAAILGLGLVLGLLGPLAILLALYLL

Nucleotide sequence of 4-1BB immune co-stimulator intracellular segment:

AAGCGCGGCCGCAAGAAGCTGCTGTACATCTTCAAGCAGCCCTTCATGC
GCCCCGTGCAGACCACCCAGGAGGAGGACGGCTGTAGCTGCCGCTTCCC
CGAGGAGGAGGAGGGCGGCTGCGAGCTG

Amino acid sequence of 4-1BB immune co-stimulator intracellular segment:

KRGRKKLLYIFKQPFMRPVQTTQEEDGCSCRFPEEEEGGCEL

Nucleotide sequence of ICOS immune co-stimulator intracellular segment:

tgttggcttacaaaaaagaagtattcatccagtgtgcacgaccctaacg
gtgaatacatgttcatgagagcagtgaacacagccaaaaaatctagact
cacagatgtgacccta

Amino acid sequence of ICOS immune co-stimulator intracellular segment:

CWLTKKKYSSSVHDPNGEYMFMRAVNTAKKSRLTDVTL

Nucleotide sequence of OX40 immune co-stimulator intracellular segment:

CGGCGCGACCAGCGCCTGCCCCCCGACGCCCACAAGCCCCCCGGCGGC
GGCAGCTTCCGCACCCCCATCCAGGAGGAGCAGGCCGACGCCCACAGC
ACCCTGGCCAAGATC

Amino acid sequence of OX40 immune co-stimulator intracellular segment:

RRDQRLPPDAHKPPGGGSFRTPIQEEQADAHSTLAKI

Nucleotide sequence of CD3 Zeta:
CGCGTGAAATTTAGCCGCAGCGCCGACGCCCCCGCCTACCAGCAGGGCC
AGAACCAGCTGTACAACGAGCTGAACCTGGGCCGCCGCGAGGAGTACGA
CGTGCTGGACAAGCGCCGGGGCCGCGACCCCGAGATGGGCGGCAAGCCC
CAGCGCCGCAAGAACCCCCAGGAGGGCCTGTACAACGAGCTGCAGAAGG
ACAAGATGGCCGAAGCCTACAGCGAGATCGGCATGAAGGGCGAGCGCCG
CCGGGGCAAGGGCCACGACGGCCTGTACCAGGGCCTGAGCACCGCCACC
AAGGACACCTACGACGCCCTGCACATGCAGGCCCTGCCCCCCCGC
Amino acid sequence of CD3 Zeta:
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

Taking the construction of a CAR against CD19 or Claudin 18.2 antigen as an example, the constructed nucleotide sequence of CAR 1 to 10 and the expressed amino acid sequence of CAR 1 to 10 with signal peptide are as follows:

Nucleotide sequence of anti-CD19 scFv:
GACATCCAGATGACACAGACTACATCCTCCCTGTCTGCCTCTCTGGGAGACAGAG
TCACCATCAGTTGCAGGGCAAGTCAGGACATTAGTAAATATTTAAATTGGTATCAGCAG
AAACCAGATGGAACTGTTAAACTCCTGATCTACCATACATCAAGATTACACTCAGGAG
TCCCATCAAGGTTCAGTGGCAGTGGGTCTGGAACAGATTATTCTCTCACCATTAGCAA
CCTGGAGCAAGAAGATATTGCCACTTACTTTTGCCAACAGGGTAATACGCTTCCGTAC
ACGTTCGGAGGGGGGACCAAGCTGGAGATCACAGGTGGCGGTGGCTCGGGCGGTGG
TGGGTCGGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCATTACCCGA
CTATGGTGTAAGCTGGATTCGCCAGCCTCCACGAAAGGGTCTGGAGTGGCTGGGAGT
AATATGGGGTAGTGAAACCACATACTATAATTCAGCTCTCAAATCCAGACTGACCATCA
TCAAGGACAACTCCAAGAGCCAAGTTTTCTTAAAAATGAACAGTCTGCAAACTGATG
ACACAGCCATTTACTACTGTGCCAAACATTATTACTACGGTGGTAGCTATGCTATGGAC
TACTGGGGCCAAGGAACCTCAGTCACCGTCTCCTCA
Amino acid sequence of anti-CD19 scFv:
DIQMTQTTSSLSASLGDRVTISCRASQDISKYLNWYQQKPDGTVKLLIYHTSRLHSG
VPSRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYAMDYWGQGT
SVTVSS
The constructed nucleotide sequence of anti CD19-CAR1
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCC
TTTACTGCAAGCGCGGCCGCAAGAAGCTGCTGTACATCTTCAAGCAGCCCTTCATGCG
CCCCGTGCAGACCACCCAGGAGGAGGACGGCTGTAGCTGCCGCTTCCCCGAGGAGG
AGGAGGGCGGCTGCGAGCTGTGTTGGCTTACAAAAAAGAAGTATTCATCCAGTGTGC
ACGACCCTAACGGTGAATACATGTTCATGAGAGCAGTGAACACAGCCAAAAAATCTA
GACTCACAGATGTGACCCTACGCGTGAAATTTAGCCGCAGCGCCGACGCCCCCGCCTA
CCAGCAGGGCCAGAACCAGCTGTACAACGAGCTGAACCTGGGCCGCCGCGAGGAGT
ACGACGTGCTGGACAAGCGCCGGGGCCGCGACCCCGAGATGGGCGGCAAGCCCCAG
CGCCGCAAGAACCCCCAGGAGGGCCTGTACAACGAGCTGCAGAAGGACAAGATGGC
CGAAGCCTACAGCGAGATCGGCATGAAGGGCGAGCGCCGCCGGGGCAAGGGCCACG
ACGGCCTGTACCAGGGCCTGAGCACCGCCACCAAGGACACCTACGACGCCCTGCACA
TGCAGGCCCTGCCCCCCCGC
Amino acid sequence of anti CD19-CAR1 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDIYIWAPLAGTCGVLLLSLVITLYCKRGRKKLLYIFKQPFMRPVQTTQEEDGCSCR
FPEEEEGGCELCWLTKKKYSSSVHDPNGEYMFMRAVNTAKKSRLTDVTLRVKFSRSADA
PAYQQGQNQLYNELNLGRREEYDVLDKRRGRDPEMGGKPQRRKNPQEGLYNELQKDK
MAEAYSEIGMKGERRRGKGHDGLYQGLSTATKDTYDALHMQALPPR
The constructed nucleotide sequence of anti CD19-CAR2:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATTTCTGG
TTACCCATAGGATGTGCAGCCTTTGTTGTAGTCTGCATTTTGGGATGCATACTTATTTGT
TGGCTTTGTTGGCTTACAAAAAAGAAGTATTCATCCAGTGTGCACGACCCTAACGGTG
AATACATGTTCATGAGAGCAGTGAACACAGCCAAAAAATCTAGACTCACAGATGTGA
CCCTACGCGTGAAATTTAGCCGCAGCGCCGACGCCCCCGCCTACCAGCAGGGCCAGA
ACCAGCTGTACAACGAGCTGAACCTGGGCCGCCGCGAGGAGTACGACGTGCTGGAC
AAGCGCCGGGGCCGCGACCCCGAGATGGGCGGCAAGCCCCAGCGCCGCAAGAACCC
CCAGGAGGGCCTGTACAACGAGCTGCAGAAGGACAAGATGGCCGAAGCCTACAGCG
AGATCGGCATGAAGGGCGAGCGCCGCCGGGGCAAGGGCCACGACGGCCTGTACCAG
GGCCTGAGCACCGCCACCAAGGACACCTACGACGCCCTGCACATGCAGGCCCTGCCC
CCCCGC
Amino acid sequence of anti CD19-CAR2 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDFWLPIGCAAFVVVCILGCILICWLCWLTKKKYSSSVHDPNGEYMFMRAVNTAK
KSRLTDVTLRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALH
MQALPPR
The constructed nucleotide sequence of anti CD19-CAR3:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCC
TTTACTGCTGTTGGCTTACAAAAAAGAAGTATTCATCCAGTGTGCACGACCCTAACGG
TGAATACATGTTCATGAGAGCAGTGAACACAGCCAAAAAATCTAGACTCACAGATGT
GACCCTAAAGCGCGGCCGCAAGAAGCTGCTGTACATCTTCAAGCAGCCCTTCATGCG
CCCCGTGCAGACCACCCAGGAGGAGGACGGCTGTAGCTGCCGCTTCCCCGAGGAGG
AGGAGGGCGGCTGCGAGCTGCGCGTGAAATTTAGCCGCAGCGCCGACGCCCCCGCCT
ACCAGCAGGGCCAGAACCAGCTGTACAACGAGCTGAACCTGGGCCGCCGCGAGGAG
TACGACGTGCTGGACAAGCGCCGGGGCCGCGACCCCGAGATGGGCGGCAAGCCCCA
GCGCCGCAAGAACCCCCAGGAGGGCCTGTACAACGAGCTGCAGAAGGACAAGATGG
CCGAAGCCTACAGCGAGATCGGCATGAAGGGCGAGCGCCGCCGGGGCAAGGGCCAC
GACGGCCTGTACCAGGGCCTGAGCACCGCCACCAAGGACACCTACGACGCCCTGCAC
ATGCAGGCCCTGCCCCCCCGC
Amino acid sequence of anti CD19-CAR3 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDIYIWAPLAGTCGVLLLSLVITLYCCWLTKKKYSSSVHDPNGEYMFMRAVNTAKK
SRLTDVTLKRGRKKLLYIFKQPFMRPVQTTQEEDGCSCRFPEEEEGGCELRVKFSRSADAP
AYQQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
The constructed nucleotide sequence of anti CD19-CAR4:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATTTCTGG
TTACCCATAGGATGTGCAGCCTTTGTTGTAGTCTGCATTTTGGGATGCATACTTATTTGT
TGGCTTAAGCGCGGCCGCAAGAAGCTGCTGTACATCTTCAAGCAGCCCTTCATGCGCC
CCGTGCAGACCACCCAGGAGGAGGACGGCTGTAGCTGCCGCTTCCCCGAGGAGGAG
GAGGGCGGCTGCGAGCTGTGTTGGCTTACAAAAAAGAAGTATTCATCCAGTGTGCAC
GACCCTAACGGTGAATACATGTTCATGAGAGCAGTGAACACAGCCAAAAAATCTAGA
CTCACAGATGTGACCCTACGCGTGAAATTTAGCCGCAGCGCCGACGCCCCCGCCTACC
AGCAGGGCCAGAACCAGCTGTACAACGAGCTGAACCTGGGCCGCCGCGAGGAGTAC
GACGTGCTGGACAAGCGCCGGGGCCGCGACCCCGAGATGGGCGGCAAGCCCCAGCG
CCGCAAGAACCCCCAGGAGGGCCTGTACAACGAGCTGCAGAAGGACAAGATGGCCG
AAGCCTACAGCGAGATCGGCATGAAGGGCGAGCGCCGCCGGGGCAAGGGCCACGAC
GGCCTGTACCAGGGCCTGAGCACCGCCACCAAGGACACCTACGACGCCCTGCACATG
CAGGCCCTGCCCCCCCGC
Amino acid sequence of anti CD19-CAR4 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDFWLPIGCAAFVVVCILGCILICWLKRGRKKLLYIFKQPFMRPVQTTQEEDGCSC
RFPEEEEGGCELCWLTKKKYSSSVHDPNGEYMFMRAVNTAKKSRLTDVTLRVKFSRSAD
APAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR
The constructed nucleotide sequence of anti CD19-CAR5:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATTTCTGG
TTACCCATAGGATGTGCAGCCTTTGTTGTAGTCTGCATTTTGGGATGCATACTTATTTGT
TGGCTTTGTTGGCTTACAAAAAAGAAGTATTCATCCAGTGTGCACGACCCTAACGGTG
AATACATGTTCATGAGAGCAGTGAACACAGCCAAAAAATCTAGACTCACAGATGTGA
CCCTAAAGCGCGGCCGCAAGAAGCTGCTGTACATCTTCAAGCAGCCCTTCATGCGCCC
CGTGCAGACCACCCAGGAGGAGGACGGCTGTAGCTGCCGCTTCCCCGAGGAGGAGG
AGGGCGGCTGCGAGCTGCGCGTGAAATTTAGCCGCAGCGCCGACGCCCCCGCCTACC
AGCAGGGCCAGAACCAGCTGTACAACGAGCTGAACCTGGGCCGCCGCGAGGAGTAC
GACGTGCTGGACAAGCGCCGGGGCCGCGACCCCGAGATGGGCGGCAAGCCCCAGCG
CCGCAAGAACCCCCAGGAGGGCCTGTACAACGAGCTGCAGAAGGACAAGATGGCCG
AAGCCTACAGCGAGATCGGCATGAAGGGCGAGCGCCGCCGGGGCAAGGGCCACGAC
GGCCTGTACCAGGGCCTGAGCACCGCCACCAAGGACACCTACGACGCCCTGCACATG
CAGGCCCTGCCCCCCCGC
Amino acid sequence of anti CD19-CAR5 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDFWLPIGCAAFVVVCILGCILICWLCWLTKKKYSSSVHDPNGEYMFMRAVNTAK
KSRLTDVTLKRGRKKLLYIFKQPFMRPVQTTQEEDGCSCRFPEEEEGGCELRVKFSRSAD
APAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR
The constructed nucleotide sequence of anti CD19-CAR6:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCC
TTTACTGCCGGCGCGACCAGCGCCTGCCCCCCGACGCCCACAAGCCCCCCGGCGGCG
GCAGCTTCCGCACCCCCATCCAGGAGGAGCAGGCCGACGCCCACAGCACCCTGGCCA
AGATCAAGCGCGGCCGCAAGAAGCTGCTGTACATCTTCAAGCAGCCCTTCATGCGCC
CCGTGCAGACCACCCAGGAGGAGGACGGCTGTAGCTGCCGCTTCCCCGAGGAGGAG
GAGGGCGGCTGCGAGCTGCGCGTGAAATTTAGCCGCAGCGCCGACGCCCCCGCCTAC
CAGCAGGGCCAGAACCAGCTGTACAACGAGCTGAACCTGGGCCGCCGCGAGGAGTA
CGACGTGCTGGACAAGCGCCGGGGCCGCGACCCCGAGATGGGCGGCAAGCCCCAGC
GCCGCAAGAACCCCCAGGAGGGCCTGTACAACGAGCTGCAGAAGGACAAGATGGCC
GAAGCCTACAGCGAGATCGGCATGAAGGGCGAGCGCCGCCGGGGCAAGGGCCACGA
CGGCCTGTACCAGGGCCTGAGCACCGCCACCAAGGACACCTACGACGCCCTGCACAT
GCAGGCCCTGCCCCCCCGC
Amino acid sequence of anti CD19-CAR6 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDIYIWAPLAGTCGVLLLSLVITLYCRRDQRLPPDAHKPPGGGSFRTPIQEEQADAH
STLAKIKRGRKKLLYIFKQPFMRPVQTTQEEDGCSCRFPEEEEGGCELRVKFSRSADAPAY
QQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPR
The constructed nucleotide sequence of anti CD19-CAR7:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATGTTGCC
GCCATCCTGGGCCTGGGCCTGGTGCTGGGGCTGCTGGGCCCCCTGGCCATCCTGCTGG
CCCTGTACCTGCTCCGGCGCGACCAGCGCCTGCCCCCCGACGCCCACAAGCCCCCCG
GCGGCGGCAGCTTCCGCACCCCCATCCAGGAGGAGCAGGCCGACGCCCACAGCACC
CTGGCCAAGATCAAGCGCGGCCGCAAGAAGCTGCTGTACATCTTCAAGCAGCCCTTC
ATGCGCCCCGTGCAGACCACCCAGGAGGAGGACGGCTGTAGCTGCCGCTTCCCCGAG
GAGGAGGAGGGCGGCTGCGAGCTGCGCGTGAAATTTAGCCGCAGCGCCGACGCCCC
CGCCTACCAGCAGGGCCAGAACCAGCTGTACAACGAGCTGAACCTGGGCCGCCGCG
AGGAGTACGACGTGCTGGACAAGCGCCGGGGCCGCGACCCCGAGATGGGCGGCAAG
CCCCAGCGCCGCAAGAACCCCCAGGAGGGCCTGTACAACGAGCTGCAGAAGGACAA
GATGGCCGAAGCCTACAGCGAGATCGGCATGAAGGGCGAGCGCCGCCGGGGCAAGG
GCCACGACGGCCTGTACCAGGGCCTGAGCACCGCCACCAAGGACACCTACGACGCCC
TGCACATGCAGGCCCTGCCCCCCCGC
Amino acid sequence of anti CD19-CAR7 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDVAAILGLGLVLGLLGPLAILLALYLLRRDQRLPPDAHKPPGGGSFRTPIQEEQAD
AHSTLAKIKRGRKKLLYIFKQPFMRPVQTTQEEDGCSCRFPEEEEGGCELRVKFSRSADAP
AYQQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
The constructed nucleotide sequence of anti CD19-CAR8:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATGTTGCC
GCCATCCTGGGCCTGGGCCTGGTGCTGGGGCTGCTGGGCCCCCTGGCCATCCTGCTGG
CCCTGTACCTGCTCAAGCGCGGCCGCAAGAAGCTGCTGTACATCTTCAAGCAGCCCTT
CATGCGCCCCGTGCAGACCACCCAGGAGGAGGACGGCTGTAGCTGCCGCTTCCCCGA
GGAGGAGGAGGGCGGCTGCGAGCTGCGGCGCGACCAGCGCCTGCCCCCCGACGCCC
ACAAGCCCCCCGGCGGCGGCAGCTTCCGCACCCCCATCCAGGAGGAGCAGGCCGAC
GCCCACAGCACCCTGGCCAAGATCCGCGTGAAATTTAGCCGCAGCGCCGACGCCCCC
GCCTACCAGCAGGGCCAGAACCAGCTGTACAACGAGCTGAACCTGGGCCGCCGCGA
GGAGTACGACGTGCTGGACAAGCGCCGGGGCCGCGACCCCGAGATGGGCGGCAAGC
CCCAGCGCCGCAAGAACCCCCAGGAGGGCCTGTACAACGAGCTGCAGAAGGACAAG
ATGGCCGAAGCCTACAGCGAGATCGGCATGAAGGGCGAGCGCCGCCGGGGCAAGGG
CCACGACGGCCTGTACCAGGGCCTGAGCACCGCCACCAAGGACACCTACGACGCCCT
GCACATGCAGGCCCTGCCCCCCCGC
Amino acid sequence of anti CD19-CAR8 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDVAAILGLGLVLGLLGPLAILLALYLLKRGRKKLLYIFKQPFMRPVQTTQEEDGCS
CRFPEEEEGGCELRRDQRLPPDAHKPPGGGSFRTPIQEEQADAHSTLAKIRVKFSRSADAP
AYQQGQNQLYNELNLGRREEYDVLDKRRGRDPEMGGKPQRRKNPQEGLYNELQKDKM
AEAYSEIGMKGERRRGKGHDGLYQGLSTATKDTYDALHMQALPPR
The constructed nucleotide sequence of anti CD19-CAR9:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCC
TTTACTGCAAGCGCGGCCGCAAGAAGCTGCTGTACATCTTCAAGCAGCCCTTCATGCG
CCCCGTGCAGACCACCCAGGAGGAGGACGGCTGTAGCTGCCGCTTCCCCGAGGAGG
AGGAGGGCGGCTGCGAGCTGCGGCGCGACCAGCGCCTGCCCCCCGACGCCCACAAG
CCCCCCGGCGGCGGCAGCTTCCGCACCCCCATCCAGGAGGAGCAGGCCGACGCCCAC
AGCACCCTGGCCAAGATCCGCGTGAAATTTAGCCGCAGCGCCGACGCCCCCGCCTAC
CAGCAGGGCCAGAACCAGCTGTACAACGAGCTGAACCTGGGCCGCCGCGAGGAGTA
CGACGTGCTGGACAAGCGCCGGGGCCGCGACCCCGAGATGGGCGGCAAGCCCCAGC
GCCGCAAGAACCCCCAGGAGGGCCTGTACAACGAGCTGCAGAAGGACAAGATGGCC
GAAGCCTACAGCGAGATCGGCATGAAGGGCGAGCGCCGCCGGGGCAAGGGCCACGA
CGGCCTGTACCAGGGCCTGAGCACCGCCACCAAGGACACCTACGACGCCCTGCACAT
GCAGGCCCTGCCCCCCCGC
Amino acid sequence of anti CD19-CAR9 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDIYIWAPLAGTCGVLLLSLVITLYCKRGRKKLLYIFKQPFMRPVQTTQEEDGCSCR
FPEEEEGGCELRRDQRLPPDAHKPPGGGSFRTPIQEEQADAHSTLAKIRVKFSRSADAPAY
QQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPR
The constructed nucleotide sequence of anti CD19-CAR10:
ATGGGCGTCAAGGTCCTGTTCGCCCTGATCTGCATCGCCGTCGCCGAGGCCGACA
TCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATC
AAGGTTCAGTGGCAGTGGGTCTGGAACAGATTATTCTCTCACCATTAGCAACCTGGAG
CAAGAAGATATTGCCACTTACTTTTGCCAACAGGGTAATACGCTTCCGTACACGTTCG
GAGGGGGGACCAAGCTGGAGATCACAGGTGGCGGTGGCTCGGGCGGTGGTGGGTCG
GGTGGCGGCGGATCTGAGGTGAAACTGCAGGAGTCAGGACCTGGCCTGGTGGCGCC
CTCACAGAGCCTGTCCGTCACATGCACTGTCTCAGGGGTCTCATTACCCGACTATGGT
GTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGG
ACAACTCCAAGAGCCAAGTTTTCTTAAAAATGAACAGTCTGCAAACTGATGACACAG
CCATTTACTACTGTGCCAAACATTATTACTACGGTGGTAGCTATGCTATGGACTACTGG
GGCCAAGGAACCTCAGTCACCGTCTCCTCAACCACGACGCCAGCGCCGCGACCACCA
ACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCC
TTTACTGCAAGCGCGGCCGCAAGAAGCTGCTGTACATCTTCAAGCAGCCCTTCATGCG
CCCCGTGCAGACCACCCAGGAGGAGGACGGCTGTAGCTGCCGCTTCCCCGAGGAGG
AGGAGGGCGGCTGCGAGCTGCGCGTGAAATTTAGCCGCAGCGCCGACGCCCCCGCCT
ACCAGCAGGGCCAGAACCAGCTGTACAACGAGCTGAACCTGGGCCGCCGCGAGGAG
TACGACGTGCTGGACAAGCGCCGGGGCCGCGACCCCGAGATGGGCGGCAAGCCCCA
GCGCCGCAAGAACCCCCAGGAGGGCCTGTACAACGAGCTGCAGAAGGACAAGATGG
CCGAAGCCTACAGCGAGATCGGCATGAAGGGCGAGCGCCGCCGGGGCAAGGGCCAC
GACGGCCTGTACCAGGGCCTGAGCACCGCCACCAAGGACACCTACGACGCCCTGCAC
ATGCAGGCCCTGCCCCCCCGC
Amino acid sequence of anti CD19-CAR10 with signal peptide:
MGVKVLFALICIAVAEADIQMTQTTSSLSASLGDRVTISCRASQDISKYLNWYQQKP
DGTVKLLIYHTSRLHSGVPSRFSGSGSGTDYSLTISNLEQEDIATYFCQQGNTLPYTFGGG
TKLEITGGGGSGGGGSGGGGSEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHY
YYGGSYAMDYWGQGTSVTVSSTTTPAPRPPTPAPTIASQPLSLRPEACRPAAGGAVHTRG
LDFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDAL
HMQALPPR
Nucleotide sequence of anti-Claudin 18.2 scFv:
GATATCGTGATGACACAGAGCCCTAGCTCTTTAACAGTGACCGCCGGCGAGAAG
GTGACCATGAGCTGCAAGAGCAGCCAGTCTTTACTGAACTCCGGCAACCAGAAGAAC
TATTTAACTTGGTACCAGCAGAAGCCCGGCCAGCCCCCTAAGCTGCTCATCTACTGGG
CCAGCACTCGTGAGAGCGGCGTGCCCGATAGATTCACTGGTTCCGGCAGCGGCACCG
ACTTTACTTTAACCATCAGCAGCGTGCAAGCCGAGGATTTAGCCGTGTACTACTGCCA
GAACGACTATAGCTACCCCTTCACCTTCGGCTCCGGCACCAAGCTGGAGATTAAGGGT
GGCGGTGGCTCGGGCGGTGGTGGGTCGGGTGGCGGCGGATCTCAAGTTCAGCTGCAG
CAACCCGGAGCCGAACTGGTGAGACCCGGAGCTAGCGTGAAGCTGTCTTGTAAGGCC
TCCGGCTATACATTTACCAGCTACTGGATCAATTGGGTGAAGCAGAGACCCGGCCAAG
GTTTAGAGTGGATCGGAAACATCTACCCCTCCGATAGCTACACCAACTACAACCAGAA
GTTCAAGGACAAGGCCACACTCACAGTGGACAAAAGCAGCAGCACCGCCTACATGC
AGCTGAGCAGCCCCACCTCCGAGGATAGCGCCGTGTACTATTGCACTCGTTCTTGGAG
GGGCAATAGCTTCGACTATTGGGGCCAAGGTACCACCCTCACCGTGAGCAGC
Amino acid sequence of anti-Claudin 18.2 scFv:
DIVMTQSPSSLTVTAGEKVTMSCKSSQSLLNSGNQKNYLTWYQQKPGQPPKLLIYW
ASTRESGVPDRFTGSGSGTDFTLTISSVQAEDLAVYYCQNDYSYPFTFGSGTKLEIKGGGG
SGGGGSGGGGSQVQLQQPGAELVRPGASVKLSCKASGYTFTSYWINWVKQRPGQGLEW
IGNIYPSDSYTNYNQKFKDKATLTVDKSSSTAYMQLSSPTSEDSAVYYCTRSWRGNSFDY
WGQGTTLTVSS
The constructed nucleotide sequence of anti Claudin 18.2-CARl:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGT
CACTGGTTATCACCCTTTACTGCAAGCGCGGCCGCAAGAAGCTGCTGTACATCTTCAA
GCAGCCCTTCATGCGCCCCGTGCAGACCACCCAGGAGGAGGACGGCTGTAGCTGCCG
CTTCCCCGAGGAGGAGGAGGGCGGCTGCGAGCTGTGTTGGCTTACAAAAAAGAAGTA
TTCATCCAGTGTGCACGACCCTAACGGTGAATACATGTTCATGAGAGCAGTGAACACA
GCCAAAAAATCTAGACTCACAGATGTGACCCTACGCGTGAAATTTAGCCGCAGCGCC
GACGCCCCCGCCTACCAGCAGGGCCAGAACCAGCTGTACAACGAGCTGAACCTGGG
CCGCCGCGAGGAGTACGACGTGCTGGACAAGCGCCGGGGCCGCGACCCCGAGATGG
GCGGCAAGCCCCAGCGCCGCAAGAACCCCCAGGAGGGCCTGTACAACGAGCTGCAG
AAGGACAAGATGGCCGAAGCCTACAGCGAGATCGGCATGAAGGGCGAGCGCCGCCG
GGGCAAGGGCCACGACGGCCTGTACCAGGGCCTGAGCACCGCCACCAAGGACACCT
ACGACGCCCTGCACATGCAGGCCCTGCCCCCCCGC
Amino acid sequence of anti Claudin 18.2-CAR1 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEE
DGCSCRFPEEEEGGCELCWLTKKKYSSSVHDPNGEYMFMRAVNTAKKSRLTDVTLRVKF
SRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
The constructed nucleotide sequence of anti Claudin 18.2-CAR2:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATTTCTGGTTACCCATAGGATGTGCAGCCTTTGTTGTAGTCTGCATTTTGG
GATGCATACTTATTTGTTGGCTTTGTTGGCTTACAAAAAAGAAGTATTCATCCAGTGTG
CACGACCCTAACGGTGAATACATGTTCATGAGAGCAGTGAACACAGCCAAAAAATCT
AGACTCACAGATGTGACCCTACGCGTGAAATTTAGCCGCAGCGCCGACGCCCCCGCCT
ACCAGCAGGGCCAGAACCAGCTGTACAACGAGCTGAACCTGGGCCGCCGCGAGGAG
TACGACGTGCTGGACAAGCGCCGGGGCCGCGACCCCGAGATGGGCGGCAAGCCCCA
GCGCCGCAAGAACCCCCAGGAGGGCCTGTACAACGAGCTGCAGAAGGACAAGATGG
CCGAAGCCTACAGCGAGATCGGCATGAAGGGCGAGCGCCGCCGGGGCAAGGGCCAC
GACGGCCTGTACCAGGGCCTGAGCACCGCCACCAAGGACACCTACGACGCCCTGCAC
ATGCAGGCCCTGCCCCCCCGC
Amino acid sequence of anti Claudin 18.2-CAR2 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDFWLPIGCAAFVVVCILGCILICWLCWLTKKKYSSSVHDPNGEYMFMR
AVNTAKKSRLTDVTLRVKFSRSADAPAYQQGQNQLYNELNLGRREEYDVLDKRRGRDPE
MGGKPQRRKNPQEGLYNELQKDKMAEAYSEIGMKGERRRGKGHDGLYQGLSTATKDT
YDALHMQALPPR
The constructed nucleotide sequence of anti Claudin 18.2-CAR3:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGT
CACTGGTTATCACCCTTTACTGCTGTTGGCTTACAAAAAAGAAGTATTCATCCAGTGTG
CACGACCCTAACGGTGAATACATGTTCATGAGAGCAGTGAACACAGCCAAAAAATCT
AGACTCACAGATGTGACCCTAAAGCGCGGCCGCAAGAAGCTGCTGTACATCTTCAAG
CAGCCCTTCATGCGCCCCGTGCAGACCACCCAGGAGGAGGACGGCTGTAGCTGCCGC
TTCCCCGAGGAGGAGGAGGGCGGCTGCGAGCTGCGCGTGAAATTTAGCCGCAGCGCC
GACGCCCCCGCCTACCAGCAGGGCCAGAACCAGCTGTACAACGAGCTGAACCTGGG
CCGCCGCGAGGAGTACGACGTGCTGGACAAGCGCCGGGGCCGCGACCCCGAGATGG
GCGGCAAGCCCCAGCGCCGCAAGAACCCCCAGGAGGGCCTGTACAACGAGCTGCAG
AAGGACAAGATGGCCGAAGCCTACAGCGAGATCGGCATGAAGGGCGAGCGCCGCCG
GGGCAAGGGCCACGACGGCCTGTACCAGGGCCTGAGCACCGCCACCAAGGACACCT
ACGACGCCCTGCACATGCAGGCCCTGCCCCCCCGC
Amino acid sequence of anti Claudin 18.2-CAR3 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDIYIWAPLAGTCGVLLLSLVITLYCCWLTKKKYSSSVHDPNGEYMFMRA
VNTAKKSRLTDVTLKRGRKKLLYIFKQPFMRPVQTTQEEDGCSCRFPEEEEGGCELRVKF
SRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
The constructed nucleotide sequence of anti Claudin 18.2-CAR4:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATTTCTGGTTACCCATAGGATGTGCAGCCTTTGTTGTAGTCTGCATTTTGG
GATGCATACTTATTTGTTGGCTTAAGCGCGGCCGCAAGAAGCTGCTGTACATCTTCAA
GCAGCCCTTCATGCGCCCCGTGCAGACCACCCAGGAGGAGGACGGCTGTAGCTGCCG
CTTCCCCGAGGAGGAGGAGGGCGGCTGCGAGCTGTGTTGGCTTACAAAAAAGAAGTA
TTCATCCAGTGTGCACGACCCTAACGGTGAATACATGTTCATGAGAGCAGTGAACACA
GCCAAAAAATCTAGACTCACAGATGTGACCCTACGCGTGAAATTTAGCCGCAGCGCC
GACGCCCCCGCCTACCAGCAGGGCCAGAACCAGCTGTACAACGAGCTGAACCTGGG
CCGCCGCGAGGAGTACGACGTGCTGGACAAGCGCCGGGGCCGCGACCCCGAGATGG
GCGGCAAGCCCCAGCGCCGCAAGAACCCCCAGGAGGGCCTGTACAACGAGCTGCAG
AAGGACAAGATGGCCGAAGCCTACAGCGAGATCGGCATGAAGGGCGAGCGCCGCCG
GGGCAAGGGCCACGACGGCCTGTACCAGGGCCTGAGCACCGCCACCAAGGACACCT
ACGACGCCCTGCACATGCAGGCCCTGCCCCCCCGC
Amino acid sequence of anti Claudin 18.2-CAR4 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDFWLPIGCAAFVVVCILGCILICWLKRGRKKLLYIFKQPFMRPVQTTQE
EDGCSCRFPEEEEGGCELCWLTKKKYSSSVHDPNGEYMFMRAVNTAKKSRLTDVTLRVK
FSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR
The constructed nucleotide sequence of anti Claudin 18.2-CAR5:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATTTCTGGTTACCCATAGGATGTGCAGCCTTTGTTGTAGTCTGCATTTTGG
GATGCATACTTATTTGTTGGCTTTGTTGGCTTACAAAAAAGAAGTATTCATCCAGTGTG
CACGACCCTAACGGTGAATACATGTTCATGAGAGCAGTGAACACAGCCAAAAAATCT
AGACTCACAGATGTGACCCTAAAGCGCGGCCGCAAGAAGCTGCTGTACATCTTCAAG
CAGCCCTTCATGCGCCCCGTGCAGACCACCCAGGAGGAGGACGGCTGTAGCTGCCGC
TTCCCCGAGGAGGAGGAGGGCGGCTGCGAGCTGCGCGTGAAATTTAGCCGCAGCGCC
GACGCCCCCGCCTACCAGCAGGGCCAGAACCAGCTGTACAACGAGCTGAACCTGGG
CCGCCGCGAGGAGTACGACGTGCTGGACAAGCGCCGGGGCCGCGACCCCGAGATGG
GCGGCAAGCCCCAGCGCCGCAAGAACCCCCAGGAGGGCCTGTACAACGAGCTGCAG
AAGGACAAGATGGCCGAAGCCTACAGCGAGATCGGCATGAAGGGCGAGCGCCGCCG
GGGCAAGGGCCACGACGGCCTGTACCAGGGCCTGAGCACCGCCACCAAGGACACCT
ACGACGCCCTGCACATGCAGGCCCTGCCCCCCCGC
Amino acid sequence of anti Claudin 18.2-CAR5 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDFWLPIGCAAFVVVCILGCILICWLCWLTKKKYSSSVHDPNGEYMFMR
AVNTAKKSRLTDVTLKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR
The constructed nucleotide sequence of anti Claudin 18.2-CAR6:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGT
CACTGGTTATCACCCTTTACTGCCGGCGCGACCAGCGCCTGCCCCCCGACGCCCACAA
GCCCCCCGGCGGCGGCAGCTTCCGCACCCCCATCCAGGAGGAGCAGGCCGACGCCC
ACAGCACCCTGGCCAAGATCAAGCGCGGCCGCAAGAAGCTGCTGTACATCTTCAAGC
AGCCCTTCATGCGCCCCGTGCAGACCACCCAGGAGGAGGACGGCTGTAGCTGCCGCT
TCCCCGAGGAGGAGGAGGGCGGCTGCGAGCTGCGCGTGAAATTTAGCCGCAGCGCC
GACGCCCCCGCCTACCAGCAGGGCCAGAACCAGCTGTACAACGAGCTGAACCTGGG
CCGCCGCGAGGAGTACGACGTGCTGGACAAGCGCCGGGGCCGCGACCCCGAGATGG
GCGGCAAGCCCCAGCGCCGCAAGAACCCCCAGGAGGGCCTGTACAACGAGCTGCAG
AAGGACAAGATGGCCGAAGCCTACAGCGAGATCGGCATGAAGGGCGAGCGCCGCCG
GGGCAAGGGCCACGACGGCCTGTACCAGGGCCTGAGCACCGCCACCAAGGACACCT
ACGACGCCCTGCACATGCAGGCCCTGCCCCCCCGC
Amino acid sequence of anti Claudin 18.2-CAR6 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDIYIWAPLAGTCGVLLLSLVITLYCRRDQRLPPDAHKPPGGGSFRTPIQEE
QADAHSTLAKIKRGRKKLLYIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYNELQ
KDKMAEAYSEIGMKGERRRGKGHDGLYQGLSTATKDTYDALHMQALPPR
The constructed nucleotide sequence of anti Claudin 18.2-CAR7:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATGTTGCCGCCATCCTGGGCCTGGGCCTGGTGCTGGGGCTGCTGGGCCCC
CTGGCCATCCTGCTGGCCCTGTACCTGCTCCGGCGCGACCAGCGCCTGCCCCCCGACG
CCCACAAGCCCCCCGGCGGCGGCAGCTTCCGCACCCCCATCCAGGAGGAGCAGGCC
GACGCCCACAGCACCCTGGCCAAGATCAAGCGCGGCCGCAAGAAGCTGCTGTACATC
TTCAAGCAGCCCTTCATGCGCCCCGTGCAGACCACCCAGGAGGAGGACGGCTGTAGC
TGCCGCTTCCCCGAGGAGGAGGAGGGCGGCTGCGAGCTGCGCGTGAAATTTAGCCGC
AGCGCCGACGCCCCCGCCTACCAGCAGGGCCAGAACCAGCTGTACAACGAGCTGAA
CCTGGGCCGCCGCGAGGAGTACGACGTGCTGGACAAGCGCCGGGGCCGCGACCCCG
AGATGGGCGGCAAGCCCCAGCGCCGCAAGAACCCCCAGGAGGGCCTGTACAACGAG
CTGCAGAAGGACAAGATGGCCGAAGCCTACAGCGAGATCGGCATGAAGGGCGAGCG
CCGCCGGGGCAAGGGCCACGACGGCCTGTACCAGGGCCTGAGCACCGCCACCAAGG
ACACCTACGACGCCCTGCACATGCAGGCCCTGCCCCCCCGC
Amino acid sequence of anti Claudin 18.2-CAR7 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDVAAILGLGLVLGLLGPLAILLALYLLRRDQRLPPDAHKPPGGGSFRTPI
QEEQADAHSTLAKIKRGRKKLLYIFKQPFMRPVQTTQEEDGCSCRFPEEEEGGCELRVKF
SRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
The constructed nucleotide sequence of anti Claudin 18.2-CAR8:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATGTTGCCGCCATCCTGGGCCTGGGCCTGGTGCTGGGGCTGCTGGGCCCC
CTGGCCATCCTGCTGGCCCTGTACCTGCTCAAGCGCGGCCGCAAGAAGCTGCTGTACA
TCTTCAAGCAGCCCTTCATGCGCCCCGTGCAGACCACCCAGGAGGAGGACGGCTGTA
GCTGCCGCTTCCCCGAGGAGGAGGAGGGCGGCTGCGAGCTGCGGCGCGACCAGCGC
CTGCCCCCCGACGCCCACAAGCCCCCCGGCGGCGGCAGCTTCCGCACCCCCATCCAG
GAGGAGCAGGCCGACGCCCACAGCACCCTGGCCAAGATCCGCGTGAAATTTAGCCGC
AGCGCCGACGCCCCCGCCTACCAGCAGGGCCAGAACCAGCTGTACAACGAGCTGAA
CCTGGGCCGCCGCGAGGAGTACGACGTGCTGGACAAGCGCCGGGGCCGCGACCCCG
AGATGGGCGGCAAGCCCCAGCGCCGCAAGAACCCCCAGGAGGGCCTGTACAACGAG
CTGCAGAAGGACAAGATGGCCGAAGCCTACAGCGAGATCGGCATGAAGGGCGAGCG
CCGCCGGGGCAAGGGCCACGACGGCCTGTACCAGGGCCTGAGCACCGCCACCAAGG
ACACCTACGACGCCCTGCACATGCAGGCCCTGCCCCCCCGC
Amino acid sequence of anti Claudin 18.2-CAR8 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDVAAILGLGLVLGLLGPLAILLALYLLKRGRKKLLYIFKQPFMRPVQTTQ
EEDGCSCRFPEEEEGGCELRRDQRLPPDAHKPPGGGSFRTPIQEEQADAHSTLAKIRVKFS
RSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPR
The constructed nucleotide sequence of anti Claudin 18.2-CAR9:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGT
CACTGGTTATCACCCTTTACTGCAAGCGCGGCCGCAAGAAGCTGCTGTACATCTTCAA
GCAGCCCTTCATGCGCCCCGTGCAGACCACCCAGGAGGAGGACGGCTGTAGCTGCCG
CTTCCCCGAGGAGGAGGAGGGCGGCTGCGAGCTGCGGCGCGACCAGCGCCTGCCCC
CCGACGCCCACAAGCCCCCCGGCGGCGGCAGCTTCCGCACCCCCATCCAGGAGGAGC
AGGCCGACGCCCACAGCACCCTGGCCAAGATCCGCGTGAAATTTAGCCGCAGCGCCG
ACGCCCCCGCCTACCAGCAGGGCCAGAACCAGCTGTACAACGAGCTGAACCTGGGCC
GCCGCGAGGAGTACGACGTGCTGGACAAGCGCCGGGGCCGCGACCCCGAGATGGGC
GGCAAGCCCCAGCGCCGCAAGAACCCCCAGGAGGGCCTGTACAACGAGCTGCAGAA
GGACAAGATGGCCGAAGCCTACAGCGAGATCGGCATGAAGGGCGAGCGCCGCCGGG
GCAAGGGCCACGACGGCCTGTACCAGGGCCTGAGCACCGCCACCAAGGACACCTAC
GACGCCCTGCACATGCAGGCCCTGCCCCCCCGC
Amino acid sequence of anti Claudin 18.2-CAR9 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEE
DGCSCRFPEEEEGGCELRRDQRLPPDAHKPPGGGSFRTPIQEEQADAHSTLAKIRVKFSRS
ADAPAYQQGQNQLYNELNLGRREEYDVLDKRRGRDPEMGGKPQRRKNPQEGLYNELQ
KDKMAEAYSEIGMKGERRRGKGHDGLYQGLSTATKDTYDALHMQALPPR
The constructed nucleotide sequence of anti Claudin 18.2-CAR10:
ATGGGCGTCAAGGTCCTGTTCGCCCTGATCTGCATCGCCGTCGCCGAGGCCGATA
TCGTGATGACACAGAGCCCTAGCTCTTTAACAGTGACCGCCGGCGAGAAGGTGACCA
TGAGCTGCAAGAGCAGCCAGTCTTTACTGAACTCCGGCAACCAGAAGAACTATTTAA
CTTGGTACCAGCAGAAGCCCGGCCAGCCCCCTAAGCTGCTCATCTACTGGGCCAGCA
CTCGTGAGAGCGGCGTGCCCGATAGATTCACTGGTTCCGGCAGCGGCACCGACTTTAC
TTTAACCATCAGCAGCGTGCAAGCCGAGGATTTAGCCGTGTACTACTGCCAGAACGAC
TATAGCTACCCCTTCACCTTCGGCTCCGGCACCAAGCTGGAGATTAAGGGTGGCGGTG
GCTCGGGCGGTGGTGGGTCGGGTGGCGGCGGATCTCAAGTTCAGCTGCAGCAACCCG
GAGCCGAACTGGTGAGACCCGGAGCTAGCGTGAAGCTGTCTTGTAAGGCCTCCGGCT
ATACATTTACCAGCTACTGGATCAATTGGGTGAAGCAGAGACCCGGCCAAGGTTTAGA
GTGGATCGGAAACATCTACCCCTCCGATAGCTACACCAACTACAACCAGAAGTTCAAG
GACAAGGCCACACTCACAGTGGACAAAAGCAGCAGCACCGCCTACATGCAGCTGAG
CAGCCCCACCTCCGAGGATAGCGCCGTGTACTATTGCACTCGTTCTTGGAGGGGCAAT
AGCTTCGACTATTGGGGCCAAGGTACCACCCTCACCGTGAGCAGCACCACGACGCCA
GCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGT
CACTGGTTATCACCCTTTACTGCAAGCGCGGCCGCAAGAAGCTGCTGTACATCTTCAA
GCAGCCCTTCATGCGCCCCGTGCAGACCACCCAGGAGGAGGACGGCTGTAGCTGCCG
CTTCCCCGAGGAGGAGGAGGGCGGCTGCGAGCTGCGCGTGAAATTTAGCCGCAGCGC
CGACGCCCCCGCCTACCAGCAGGGCCAGAACCAGCTGTACAACGAGCTGAACCTGG
GCCGCCGCGAGGAGTACGACGTGCTGGACAAGCGCCGGGGCCGCGACCCCGAGATG
GGCGGCAAGCCCCAGCGCCGCAAGAACCCCCAGGAGGGCCTGTACAACGAGCTGCA
GAAGGACAAGATGGCCGAAGCCTACAGCGAGATCGGCATGAAGGGCGAGCGCCGCC
GGGGCAAGGGCCACGACGGCCTGTACCAGGGCCTGAGCACCGCCACCAAGGACACC
TACGACGCCCTGCACATGCAGGCCCTGCCCCCCCGC
Amino acid sequence of anti Claudin 18.2-CAR10 with signal peptide:
MGVKVLFALICIAVAEADIVMTQSPSSLTVTAGEKVTMSCKSSQSLLNSGNQKNYLT
WYQQKPGQPPKLLIYWASTRESGVPDRFTGSGSGTDFTLTISSVQAEDLAVYYCQNDYSY
PFTFGSGTKLEIKGGGGSGGGGSGGGGSQVQLQQPGAELVRPGASVKLSCKASGYTFTS
YWINWVKQRPGQGLEWIGNIYPSDSYTNYNQKFKDKATLTVDKSSSTAYMQLSSPTSED
SAVYYCTRSWRGNSFDYWGQGTTLTVSSTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEE
DGCSCRFPEEEEGGCELRVKFSRSADAPAYQQGQNQLYNELNLGRREEYDVLDKRRGRD
PEMGGKPQRRKNPQEGLYNELQKDKMAEAYSEIGMKGERRRGKGHDGLYQGLSTATKD
TYDALHMQALPPR

The designed nucleotide sequence encoding various CAR structures synthesized by GENEWIZ were cut by BamH I and Sal I and cloned into lentiviral vector LV-CAR (constructing by our company), and transferred into stb13 competent cells for amplification, and plasmids were extracted for standby.

2. Lentivirus packaging of anti-CD19 CAR or anti claudin 18.2 CAR with different structures

a. 293T cells were inoculated into a 6-well plate containing 2 mL lentivirus packing medium at a density of 3×10⁶ cells/well and cultured overnight in a 37° C. 5% CO₂ incubator for standby.

b. Lentivirus packaging was performed when the cell density reached about 95% by observing, the lentivirus packaging system was configured according to the configuration system listed in Table 2 below.

TABLE 2

| components | Volume | |
|---|---|---|
| A liquid | | |
| Opti-MEM serum reduced media | 250 | μL |
| Lipofectamine 3000 transfection reagent | 7 | μL |
| B liquid | | |
| Opti-MEM serum reduced media | 250 | μL |
| P3000-Enhancer reagent | 6 | μL |
| Virapower lentivirus packaging mixture | 2.25 | μL |
| (1 μg/μL) | | |
| LV-CAR expression vector | 0.75 | μg |

A liquid configured in advance was transferred to B liquid, the mixture was mixed well, and then stood at room temperature for 20 min for standby.

c. 1 mL medium was removed per well of the 6-well plate inoculated with 293T cells, after removing the medium, 500 μL A+B mixture was added into each well, after mixing well, the plate was placed into a 37° C. 5% CO₂ incubator for 6 hours, and then the medium in the 6-well plate was replaced with lentivirus packaging medium, the 6-well plate was placed into the incubator for additional 52 hours, the culture supernatant was collected and concentrated 20 times (100 μL/tube), and stored at −80° C. for standby.

### Example 2

1. T cell activation and lentivirus infection

2 mL of CD3⁺ positive cells (Vitality 98.5%, living cells 5E+06) was added into a 15 mL centrifuge tube containing 5 mL AIM-V, the cells was collected by centrifugation at 500 g for 5 min and the supernatant was discarded, the collected cells were resuspended in 10 mL AIM-V culture medium (containing 20 IU/mL of IL-2) and the cells suspension was removed to a 10 cm petri dish for culture. 180 μL CD3/28 magnetic beads were washed by adding 4 mL of buffer, which was put on a magnetic stand for 2 min, and the supernatant was removed, and then resuspended in 180 μL of AIM-V medium (containing 20 IU/mL of IL-2). The magnetic beads suspension was added to the cells suspension, and which were mixed well and placed in an incubator for culture. Viral transfection was performed after cultivation for 24 hours.

2. The above cells mixture was added into a 15 mL centrifuge tube, and the tube was placed on a magnetic stand for 2 min, the liquid containing the cells of the cells mixture was pipetted into another 15 mL clean centrifuge tube. 10 μL of the liquid containing the cells was mixed with 10 μL AO/PI staining solution, and the mixture was added to a cell counting chamber, vitality 98.41%, living cell density 4.01E+06.3 mL of the liquid containing the cells was centrifuged at 500 g for 5 min and the supernatant was discarded. The remaining cells were resuspended in 1.2 mL AIM-V culture medium (containing 20 IU/mL of IL-2) at a concentration of 10⁷ cells/mL.

3. Viral infection: the activated T cells were infected in a 24-well plate according to the conditions shown in Table 3 below. After the cells and virus were mixed, they were allowed to stand at room temperature for 15 minutes, and then placed in a cell incubator for culture. The cells were collected into a 1.5 mL centrifuge tube at 24 hours after infection by centrifugation at 500 g for 5 min, the collected cells were resuspended in 1 mL AIM-V culture medium (containing 20 IU/mL of IL-2). The cells were passaged at a cell density of 0.5 E+06. When the cell density of the cells reached 5 E+06 to 1 E+07, the cells were passaged. After continuous culture for 2 days, the CAR-T positive rate was detected by flow cytometry.

TABLE 3

| | The liquid containing | |
|---|---|---|
| Lentivirus name | cells (10⁶ cells) | The liquid containing virus |
| CD3⁺ (Negative | 100 μL | 100 μL Culture medium |
| control group) | | |
| 1 | | 100 μL virus |
| 2 | | |
| 3 | | |
| 4 | | |
| 5 | | |
| 6 | | |
| 7 | | |
| 8 | | |
| 9 | | |
| 10 | | |
| (Second-generation | | |
| CAR-T control) | | |

4. CAR-T cells of different groups were collected, and each group has about 10⁶ cells, accounting for all one T cells negative control and ten experimental groups.

5. The cells were collected by centrifugation at 500 g for 5 min, the supernatant was discarded; the cells were resuspended in 500 μL PBS, and the suspension were centrifuged at 500 g for 5 min, the procedure was repeated twice, the supernatant was discarded. The cells were resuspended by adding 200 μL PBS as the negative control group, and stored at 4° C.

6. The experimental group was provided by the procedure comprising resuspending the cells in 500 μL PBS, adding 1 μL Protein L and incubating for 30 minutes at 4° C. in the dark; then collecting the cells by centrifugation at 500 g and 4° C. for 5 min, discarding the supernatant; and then resuspending the cells in 500 μL PBS, collecting the cells by centrifugation at 500 g and 4° C. for 5 min, discarding the supernatant, repeating the step three times; at last, resuspending the cells in 200 μL PBS for flow cytometry.

Results:

1. The CAR positive rates with different structures are significantly different under the infection system having the same amount of virus. The second-generation CAR structure (CAR10) is shorter than CAR 1 to 9 sequence and has a higher positive rate;

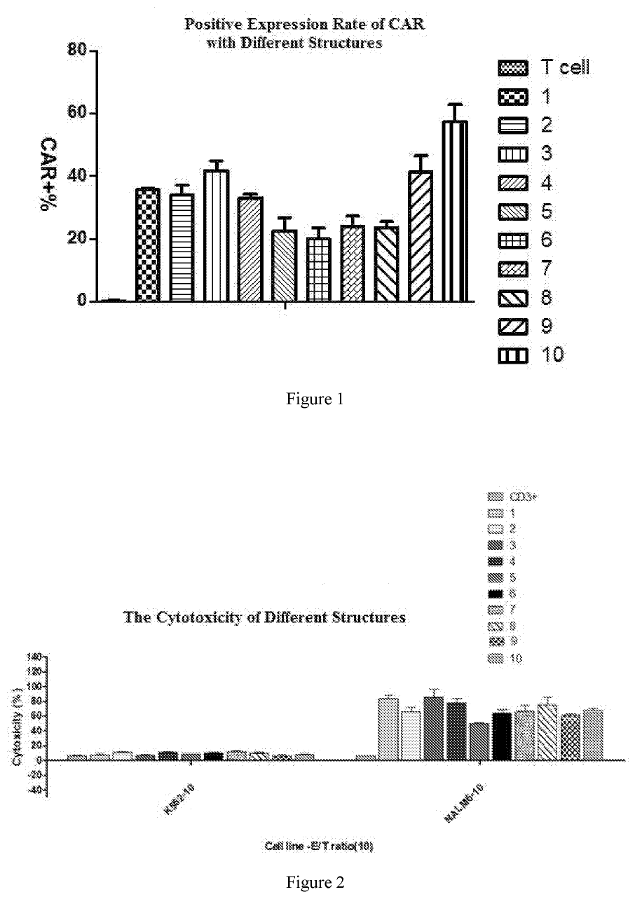

2. There is a big difference among CAR positive rates of CAR 1 to 9 structures, in which CAR 3 (CAR positive rate 41.8%) and CAR 9 (CAR positive rate 41.4%) structures have the highest positive rate (CD19 CAR results are illustrated in FIG. 1);

3. The same infection system leads to different CAR positive rate results, it indicates that the CAR structures can affect the lentiviral packaging titer. In contrast, the lentiviral titers of CAR 3 and CAR 9 structures packaging are the highest in the CAR1 to 9.

### Example 3 Detection of the Cytotoxicity of CD 19 CAR-T with Different Structures

1. Cell Culture

Effector-target cells (NALM-6/K562) were cultured in 1640/IMDM+10% FBS medium in an incubator at 37° C. and 5% carbon dioxide. Suspended cells: observing the cells using an inverted microscope once a day, and changing the culture medium every 2 to 4 days. The cells were passaged before the cell density in the flask reached 3 E+06.

2. Treatment of Effector Cells (CD 19 CAR-T with Different Structures)

The CAR-T cells with different structures were collected in a 15 mL centrifuge tube, and centrifuged at 500 g for 5 min. The supernatant was discarded, and the cells was washed by adding 4 mL of 0.01M PBS, centrifuged, and the supernatant was discarded, the cells was resuspended in an appropriate amount of AIM-V medium to make the cell density of about 10⁷cells per mL. Cells density and viability were determined.

3. Pretreatment of Target Cells

1) Target cells labeling: target cells were collected in the logarithmic growth phase and washed once with 0.01M PBS. The mixture of target cells was centrifuged at 500 g for 5 min, the supernatant was discarded; and then the target cells was resuspended in a complete medium to get a suspension with a density of 1 E+06 cells per mL. An appropriate amount of the suspension was taken and centrifuged to discard the supernatant, CFSE liquid (prepared from stock solution and preheated PBS at a ratio of 1:500) was added and mixed well to keep a final density of 1 E+06 cells per mL. The cells were incubated at 37° C., 5% CO₂ in the dark for 20 min.

2) 5 volumes of the complete medium were added and incubated for 5 minutes.

3) the mixture was centrifuged at 500 g for 5 min, then the supernatant was discarded.

4) the cells were resuspended in a pre-warmed complete medium, and the subsequent experiments were performed after which was placed at room temperature for 10 minutes.

4. Co-Incubation of Effector-Target Cells

1) NALM6⁺ effector cells group, K562⁺ effector cells group were set according to the total effector cells:target cells=10:1. Target cells and effector cells were co-incubated for 4 h, the cells were collected for flow cytometry.

Results:

1. The 10 designed CD 19 CAR-T structures all have specific killing effect on NALM6 (CD19 positive tumor cell line), but no obvious killing effect on negative cell K562 (CD19 negative cell line) (results as shown in FIG. 2);

2. Compared with the second-generation CAR-T (No. 10 CAR-T) currently in use, the specific killing of Nos. 1, 3, and 4 in the designed Nos. 1 to 9 CAR-T is significantly stronger than that of the second-generation CAR-T (shown in FIG. 2);

3. The specific killing of Nos. 2, 6, 7, 8 and 9 in the designed Nos. 1 to 9 CAR-T is comparable to that of the second-generation CAR-T (shown in FIG. 2);

4. The specific killing of No. 5 in the designed Nos. 1 to 9 CAR-T is weaker than that of the second-generation CAR-T (shown in FIG. 2);

5. The non-specific killing of No. 3 and No. 9 CAR-T is the weakest among the designed Nos. 1 to 9 CAR-T, which can reduce off-target and cellular inflammatory factor response, and improve the safety of CAR-T treatment in clinical application.

### Example 4 Cytokines Release Test of CAR-T with Different Structures in Cytotoxicity Experiment

1. the supernatant was collected after incubation of 24 h in the cytotoxicity test and diluted 5 times for use;

2. the factor content in the sample was detected by using a CBA factor detection kit (Art. No. 551809). 25 μL diluted sample+25 μL mixed magnetic beads+25 μL PE detection reagent were added to each test well. After mixing well, which was incubated at room temperature for 3 h. The mixture was washed with 200 μL PBS 3 times, and resuspended in 100 μL PBS;

3. Flow cytometry and data analysis using FCAP software.

Results:

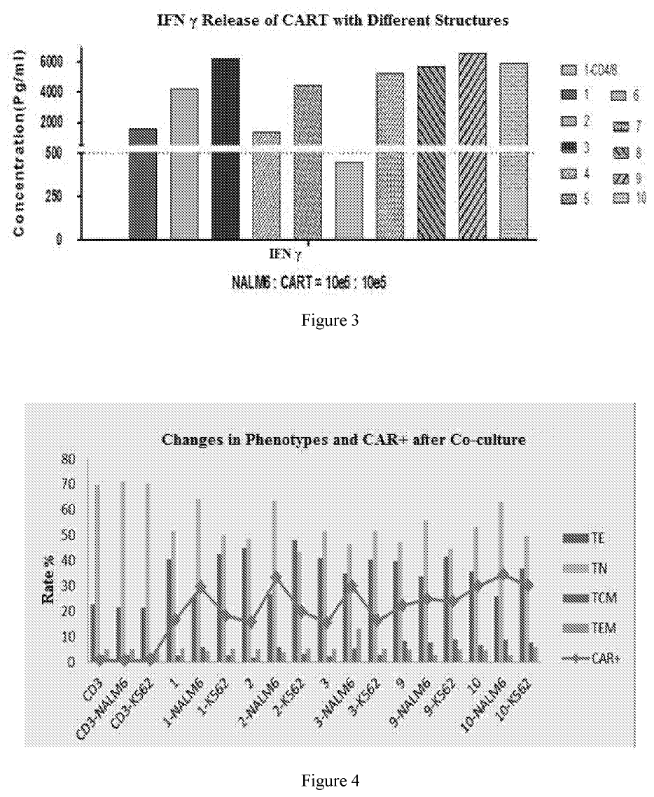

1. CAR-T with different structures all have obvious INF-γ release, and there are obvious differences between different structures, No. 3 and No. 9 have strong release (wherein the result of CD19 CAR-T as shown in FIG. 3);

2. The release of INF-γ has a certain correlation with the cytotoxicity of CAR-T. CAR-T with stronger cytotoxicity has a higher release of INF-γ;

Based on the above results of CAR⁺ positive rate, cytotoxicity experiments and the related factors release, No. 3 CAR-T (namely CAR-T 3) performed best.

### Example 5 Changes of Phenotype and CAR Positive Rate after Co-Incubation of CD 19 CAR-T with Different Structures

1. 1 mL each of CAR-T cells with different structures and CD3+ cells were collected by centrifugation at 500 g for 5 min, and the cells were resuspended in 500 μL stain buffer and then transferred to a 96-well U-shaped plate, which was then centrifuged at 500 g for 5 min to collect the cells. The cells collected were washed and resuspended three times according to the above conditions, lastly resuspended in 500 μL stain buffer.

2. 1 μL of protein L (FITC) was added to the corresponding detection well and mixed well, then equal amount of staining buffer was added to the corresponding negative control well, and reacted at 4° C. for 45 min. After the reaction was completed, the cells were washed and resuspended according to step 1.

3. 5 μL each of Anti-CD3 (APC-cy7), Anti-CD4 (BV510), Anti-CD8a (Percp-cy5.5), Anti-HuCD45RA (BV421), and 20 μL of Anti-CD197(PE) were added to the resuspended cell detection well, to the remaining wells was added equal volume of staining buffer, and reacted at 4° C. for 30 min.

4. After the reaction was completed, the cells were washed according to step 1 (adding 500 μL of staining buffer during washing) and the cells were collected. Finally, 200 μL of staining buffer was added to each well to resuspend the cells for use.

5. Preparation of tone compensation samples: 200 μL of stain buffer was added to the corresponding 96-well U-shaped plate, and to each well were added a drop of BD comp-beads negative control (mixed vigorously before use) and a drop of BD comp-beads anti-mouse Ig k pellet (mixed well before use), and 5 μL of detection antibody (CD3/CD4/CD8/CD45RA/CD4FITC) were added to the corresponding wells; to another well were added a drop of BD comp-beads negative control (mixed well before use) and a drop of BD comp-beads anti-rat Ig k pellet (mixed vigorously before use), then 20 μL of anti-CD197 antibody was added, the plate was incubated at 4° C. for 30 min, the pellet was washed and resuspended according to step 1 for standby.

6. The results of flow cytometry were as shown in FIG. 4, wherein abbreviations are TE (Effector T cell), TN (naive T cell), T_{CM} (Central memory T cell), T_{EM} (Effect memory T Cell, Effector memory T cell).

Results:

1. CAR-T 1, 2, and 3 all showed a significant increase in CAR⁺ positive rate after stimulation of tumor cells (NALM-6), no significant increase in other structures; there was no significant change in CAR⁺ positive rate when cultured alone or stimulated by unrelated tumor cells, it can form a mechanism similar to immune response, which is more conducive to CAR-T cytotoxicity;

2. After co-incubation of CAR-T 1, 2 or 3 with tumor cells, T_{CM} had a significant increase, and for the other CAR-T, T_{CM} had little change.

3. For CAR-T 3, T_{CM} increased, and T_{EM} also had a significant increase;

4. Compared with the other CAR-T, the increased radio of T_{CM} and T_{EM} after co-incubation of CAR-T 3 with tumor cells is beneficial to the persistence of the cytotoxicity of CAR-T;

5. ICOS structure as a co-stimulator can increase the ratio of T_{CM} and T_{EM}.

### Example 6 Detection of Self-Releasing Factor of CD 19 CAR-T

1. the supernatant after the culture of K562 and CAR-T for 24 h was collected, and diluted 5 times for use.

2. the factor content in the sample was detected by using a CBA factor detection kit (Art. No. 551809). 25 μL diluted sample+25 μL mixed magnetic beads+25 μL PE detection reagent were added to each test well. After mixing well, the plate was incubated at room temperature for 3 h. The mixture was washed with 200 μL PBS once, and resuspended in 100 μL PBS.

3. Flow cytometry and data analysis using FCAP software.

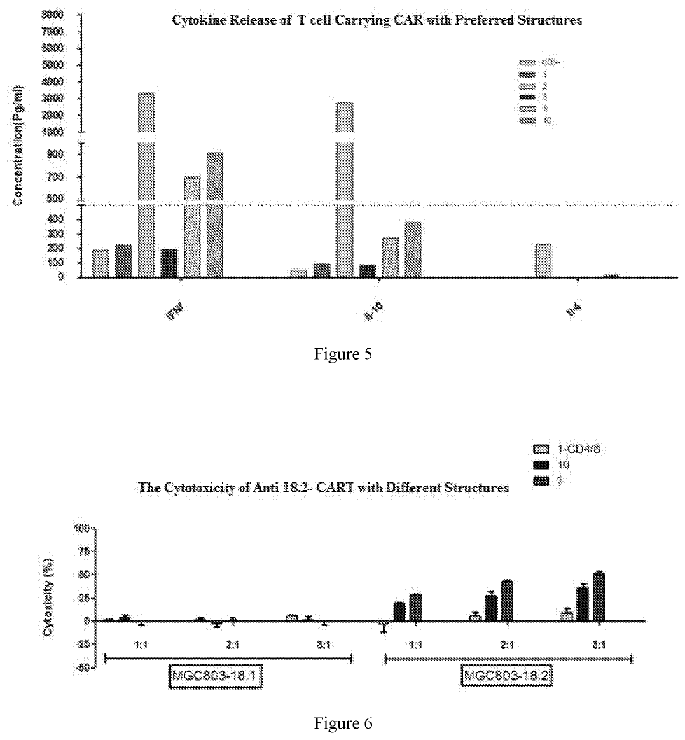

The results were shown as FIG. 5:

when CAR-T was co-incubated with unrelated tumor cells, there was a significant difference in cytokine release among different CAR-T, CAR-T 1 and 3 structures had lower INF-γ and IL10 release, and CAR-T 2, 9, 10 had higher INF-γ and IL10 release, of which CAR-T 2 has the highest release;

there is no obvious release of IL4 except CAR-T 2 structure;

when CAR-T with different structures are co-incubated with K562 cells, the release amount of factor reflects the state of CAR-T self-activation. The higher the factor release, it indicates that the CAR-T structure is in a self-activated state, which may cause a higher apoptosis factors storm, and also, the continuous self-activated state may cause cell failure, and reduce the persistence of CAR-T.

Considering comprehensively the cytotoxicity, factor release, phenotypic change and positive rate change, CAR-T 3 structure is preferred.

### Example 7 Cytotoxicity Detection of Anti-Claudin 18.2 CAR-T (CAR-T 3)

1. Cell Culture

Effective target cells (MGC-803-18.2, namely self-constructed claudin 18.2 high-expression cell line/MGC-803-18.1, namely self-constructed claudin 18.1 high-expression cell line) were cultured by using 1640/IMDM+10% FBS medium in a 5% carbon dioxide incubator at 37° C. Adherent cells: the cells were observed by using an inverted microscope once a day, and passaged every 2 days. The cells were passaged before the cell density in the flask reached 95% (covering the entire bottom of the plate by observing under the microscope).

2. Treatment of Effector Cells (Anti-Claudin 18.2 CAR-T)

The anti-Claudin 18.2 CAR-T cells were collected in a 15 mL centrifuge tube, and centrifuged at 500 g for 5 min. The supernatant was discarded, and the cells was washed by adding 4 mL of 0.01M PBS, centrifuged, and the supernatant was discarded, the cells was resuspended in an appropriate amount of AIM-V medium to make the cell density of about 10⁷cells per mL. Cells density and viability were determined.

3. Pretreatment of Target Cells

1) Target cells labeling: target cells were collected in the logarithmic growth phase and washed once with 0.01M PBS. The mixture of target cells was centrifuged at 500 g for 5 min, the supernatant was discarded; and then the target cells was resuspended in a complete medium to get a suspension with a density of 1×10⁶cells per mL. An appropriate amount of the suspension was taken and centrifuged to discard the supernatant, CFSE liquid (prepared from stock solution and preheated PBS at a ratio of 1:500) was added and mixed well to keep a final density of 1×10⁶cells per mL. The cells were incubated at 37° C., 5% CO₂ in the dark for 20 min.

2) 5 volumes of the complete medium were added and incubated for 5 minutes.

3) the mixture was centrifuged at 500 g for 5 min, the supernatant was discarded.

4) the cells were resuspended in a pre-warmed complete medium, and the subsequent experiments were performed after which was placed at room temperature for 10 minutes.

4. Co-incubation of effector-target cells

1) Both MGC-803-18.1 effector cells group and MGC-803-18.2 effector cells group were set according to the ratio that total effector cells:target cells=1:1, 2:1, 3:1. Target cells and effector cells were co-incubated for 4 h, then the cells were collected for flow cytometry.

The results were as shown in FIG. 6:

compared with the current second-generation CAR-T (CAR-T 10), the specific killing of CAR-T 3 is significantly stronger than that of the second-generation CAR-T; CAR-T 3 has a weak non-specific killing, it can reduce off-target and cellular inflammatory factor responses, and improve the safety of CAR-T treatment in clinical applications.

### Example 8 Changes of Phenotype and CAR Positive Rate after Co-Incubation of Anti-Claudin 18.2 CAR-T with Different Structures

1. 1 mL each of co-incubation cells of anti-Claudin 18.2 CAR-T cells and different target cells (treated in Example 7), which were centrifuged at 500 g for 5 min to collect the cells, the cells were resuspended in 500 μL stain buffer and then transferred to a 96-well U-shaped plate, which was centrifuged at 500 g for 5 min to collect the cells, the cells were washed and resuspended three times according to the above conditions, lastly resuspended in 500 μL stain buffer.

2. 1 μL of protein L (FITC) was added to the corresponding detection well and mixed well, then equal amount of staining buffer was added to the corresponding negative control well, and reacted at 4° C. for 45 min. After the reaction was completed, the cells were washed and resuspended according to step 1.

3. 5 μL each of Anti-CD3 (APC-cy7), Anti-CD4 (BV510), Anti-CD8a (Percp-cy5.5), Anti-HuCD45RA (BV421), and 20 μL of Anti-CD197(PE) were added to the resuspended cell detection well, to the remaining wells was added equal volume of staining buffer, and reacted at 4° C. for 30 min.

4. After the reaction was completed, the cells were washed according to step 1 (adding 500 μL of staining buffer during washing) and the cells were collected. Finally, 200 μL of staining buffer was added to each well to resuspend the cells for use.

5. Preparation of tone compensation samples: 200 μL of stain buffer was added to the corresponding 96-well U-shaped plate, and to each well were added a drop of BD comp-beads negative control (mixed vigorously before use) and a drop of BD comp-beads anti-mouse Ig k pellet (mixed well before use), and 5 μL of detection antibody (CD3/CD4/CD8/CD45RA/CD4FITC) were added to the corresponding wells; to another well were added a drop of BD comp-beads negative control (mixed well before use) and a drop of BD comp-beads anti-rat Ig κ pellet (mixed vigorously before use), then 20 μL of anti-CD197 antibody was added, the plate was incubated at 4° C. for 30 min, the pellet was washed and resuspended according to step 1 for standby.

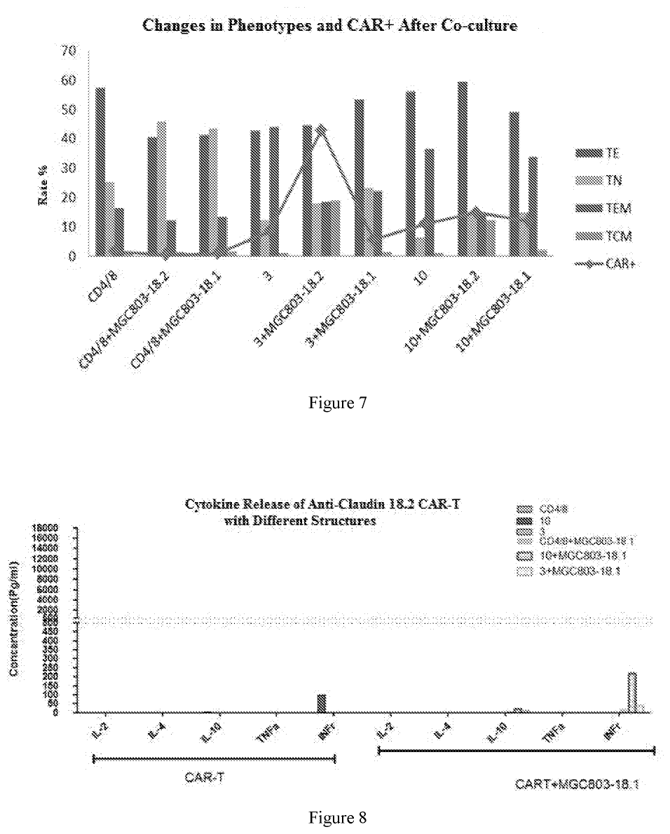

6. The flow cytometry results were as shown in FIG. 7.

1). CAR-T 3 showed a significant increase in CAR⁺ positive rate after stimulation of tumor cells (MGC803-18.2), no significant increase in other structures; there was no significant change in CAR⁺ positive rate when cultured alone or stimulated by unrelated tumor cells, so it can form a mechanism similar to immune stimulation, which is more conducive to CAR-T cytotoxicity;

2). After co-culture of CAR-T 3 with tumor cells, T_{CM} had a significant increase, and for the other CAR-T, T_{CM} had little change.

3). For CAR-T 3, T_{CM} increased, and T_{EM} also had a significant increase;

4). Compared with the other CAR-T, the increased radio of T_{CM} and T_{EM} after co-culture of CAR-T 3 with tumor cells is beneficial to the persistence of the cytotoxicity of CAR-T;

5). ICOS structure as a co-stimulator can increase the ratio of T_{CM} and T_{EM}.

### Example 9 Detection of Self-Releasing Factor of Anti-Claudin 18.2 CAR-T

1. the supernatant of anti-Claudin 18.2 CAR-T culture alone and the supernatant of MGC-803-18.1 and anti-Claudin 18.2 CAR-T co-culture for 24 h were respectively collected and diluted 5 times for use.

2. the factor content in the sample was detected by using a CBA factor detection kit (Art. No. 551809). 25 μL diluted sample+25 μL mixed magnetic beads+25 μL PE detection reagent were added to each test well. After mixing well, the plate was incubated at room temperature for 3 h. The mixture was washed with 200 μL PBS once, and resuspended in 100 μL PBS.

3. Flow cytometry and data analysis using FCAP software.

The results were as shown in FIG. 8. CAR-T 3 structure has lower INF-r and IL10 release.

Reference throughout this specification to “an embodiment”, “one embodiment”, “some embodiments”, “example”, “specific examples,” or “some examples” means that a particular feature, structure, material, or characteristic described in connection with the embodiment or example is included in at least one embodiment or example of the present disclosure. In the present specification, the schematic representation of the above terms is not necessarily directed to the same embodiment. in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A chimeric antigen receptor, wherein the chimeric antigen receptor comprises
an extracellular domain, wherein the extracellular domain includes a heavy chain variable region, a light chain variable region of a single chain antibody fragment and CD8 hinge region, the single chain antibody fragment specifically recognizes antigen;
a transmembrane domain, wherein the transmembrane domain includes an immune co-stimulator transmembrane domain; and
an intracellular domain, wherein the intracellular domain includes an immune co-stimulator intracellular segment and CD3ζ chain.

2. The chimeric antigen receptor of claim 1, wherein the immune co-stimulator transmembrane domain is CD8 transmembrane domain; the immune co-stimulator intracellular segment is 4-1BB intracellular segment and ICOS or OX-40 intracellular segment.

3. The chimeric antigen receptor of claim 1, wherein the immune co-stimulator transmembrane domain is ICOS transmembrane domain; the immune co-stimulator intracellular segment is 4-1BB intracellular segment and ICOS intracellular segment.

4. The chimeric antigen receptor of claim 1, wherein the immune co-stimulator transmembrane domain is OX40 transmembrane domain; the immune co-stimulator intracellular segment is 4-1BB intracellular segment and OX40 intracellular segment.

5. The chimeric antigen receptor of claim 1, wherein the immune co-stimulator intracellular segment is 4-1BB intracellular segment and ICOS intracellular segment, the N-terminus of the ICOS intracellular segment is linked to the C-terminus of the CD8 transmembrane domain, the C-terminus of the ICOS intracellular segment is linked to the N-terminus of the 4-1BB intracellular segment, the C-terminus of the 4-1BB intracellular segment is linked to the N-terminus of the CD3ζ chain.

6. The chimeric antigen receptor of claim 1, wherein the immune co-stimulator intracellular segment is 4-1BB intracellular segment and OX-40 intracellular segment, the N-terminus of the OX-40 intracellular segment is linked to the C-terminus of the CD8 transmembrane domain, the C-terminus of the OX-40 intracellular segment is linked to the N-terminus of the 4-1BB intracellular segment, the C-terminus of the 4-1BB intracellular segment is linked to the N-terminus of the CD3ζ chain.

7. The chimeric antigen receptor of claim 1, wherein the N-terminus of the 4-1BB intracellular segment is linked to the C-terminus of the ICOS transmembrane domain, the C-terminus of the 4-1BB intracellular segment is linked to the N-terminus of the ICOS intracellular segment, the C-terminus of the ICOS intracellular segment is linked to the N-terminus of the CD3ζ chain.

8. The chimeric antigen receptor of claim 1, wherein the immune co-stimulator transmembrane domain connected with the immune co-stimulator intracellular segment has any one amino acid sequence of SEQ ID NO:1 to 6.

9. The chimeric antigen receptor of claim 1, wherein the antigen comprises at least one selected from CD19, CD20, CD123, GPC3, MUC-1, GD2, BCMA, HER2, EGFR, VEGFR, cMet, MSLN, EGFRvIII and Claudin 18.2.

10. A construct, wherein the construct comprises a nucleic acid molecule encoding the chimeric antigen receptor of claim 1.

11. The construct of claim 10 further comprising a promoter, wherein the promoter is operably connected with the nucleic acid molecule; and
wherein the promoter comprises at least one selected from CMV, EF-1, ESV.

12. (canceled)

13. The construct of claim 10, wherein the construct is a non-pathogenic virus; and
wherein the virus is selected from retroviruses, lentiviruses and adenovirus related viruses.

14. (canceled)

15. A lentivirus, wherein the lentivirus carries a nucleic acid molecule having a nucleotide sequence of SEQ ID NO:7 to 12.

16. A transgenic lymphocyte, wherein the transgenic lymphocyte expresses the chimeric antigen receptor of claim 1.

17. (canceled)

18. (canceled)

19. The transgenic lymphocyte of claim 16, wherein the lymphocyte is a natural killer T cell.

20. A preparation method of a transgenic lymphocyte comprising
introducing the construct of claim 10 into a lymphocyte.

21. A therapeutic composition for treating cancer comprising
the construct of claim 10;
wherein the cancer comprises at least one selected from a hematopoietic malignancy, a gastrointestinal cancer, a glioma, a lung cancer, a liver cancer, a pancreatic cancer.

22. (canceled)

23. A method of improving the safety, effectiveness or persistence of lymphocyte therapy comprising expressing the chimeric antigen receptor of claim 1 by using the lymphocyte.

24. (canceled)

25. (canceled)
